(19) United States
(12) Patent Application Publication
(10) Pub. No.: US 2016/0007864 A1
(43) Pub. Date: Jan. 14, 2016

(54) SYSTEM AND METHOD FOR LACTIC THRESHOLD AND ENTRAINMENT DETECTION

(71) Applicant: Kadence Lab, LLC, Atlanta, GA (US)

(72) Inventors: John E. Scharf, Atlanta, GA (US); James Cooke, Atlanta, GA (US)

(73) Assignee: KADENCE LAB, LLC, Atlanta, GA (US)

(21) Appl. No.: 14/797,038

(22) Filed: Jul. 10, 2015

Related U.S. Application Data
(60) Provisional application No. 62/023,190, filed on Jul. 11, 2014.

(51) Int. Cl. A61B 5/0205; A61B 5/04; A61B 5/00

(52) U.S. Cl. CPC A61B 5/0205; A61B 5/0006; A61B 5/7278; A61B 5/04012; A61B 2503/10; A61B 5/02405

(57) ABSTRACT

A wearable lactic threshold and entrainment exercise device (LTEExD) is described herein. LTEExD may assist subjects in improving athletic performance. Users adjusting their exercise regimen in real-time with the assistance of LTEExD often exercise more efficiently and perceive exertional status as feeling like a ‘second wind’. LTEExD collects physiologic data, and uses a signal analyzer to convert the data from the time domain to the frequency domain primarily utilizing fast fourier transform based spectral analysis to accurately determine certain physical variables. Variables are determined, integrated, and compared by the signal analyzer to determine whether lactic threshold has been reached and/or entrainment has occurred. LTEExD may wirelessly transmit variables to a remote display, where an observer is provided real-time feedback to self-guide adjustments to an exercise regimen to improve efficiency, perceive a ‘second wind’, improve sprint and endurance fitness levels, and improve overall competitive athletic ability.

### CROSS-REFERENCE TO RELATED APPLICATIONS

This application claim priority from U.S. Provisional Application No. 62/023,190 filed on Jul. 11, 2014, entitled “Wearable Lactic Threshold and Entrainment Exercise Device,” the entire content of which is hereby incorporated by reference in its entirety.

### TECHNICAL FIELD

This disclosure relates generally to wearable exercise devices. More specifically, the disclosure provides systems and methods for detecting lactic threshold and cardio-respiro-loco-motor synchronization to improve athletic performance in the field.

### SUMMARY

Systems and methods described herein include the design and use of a wearable lactic threshold and entrainment exercise device (LTEExD). In some cases, the devices can be or act as a multi-faceted tool to assist subjects in improving athletic performance. Users adjusting their athletic and/or exercise regimen in real-time with the assistance of the LTEExD often exercise more efficiently and perceive an exertional status as feeling like a ‘second wind’. In its more detailed application, the LTEExD is a smart device that may determine, integrate, compare, evaluate, and display heart rate, heart rate variability, respiratory rate, arterial hemoglobin oxygen saturation, lactic threshold, exercise cadence, and cardio-respiro-loco-motor synchronization (entrainment) data. Without being limited thereto, the athletic or exercise with which the devices can be used and the methods utilized, can include, for example, running, swimming, cycling, cross country skiing, skating, and other endurance events and sports. The devices can be used for football, soccer and basketball training, for example, as well as other sports. The devices can be used for hiking, trekking and backpacking, in water diving, sky diving, and other activities where oxygen availability and usage may be important for safety and/or performance.

Systems and methods described herein provide a portable, low-power, wireless, real-time device, with signal analyzer. As described herein, embodiments may analyze, using fast Fourier transform (FFT) based spectral analysis, lactic threshold and entrainment in the field, under heavy exercise, including training and competition conditions, and telemeter the variable results to an observer with remote display.

A non-limiting example of a system includes a pulse oximeter, an accelerometer, and a signal analyzer. The signal analyzer may receive a motion signal from the accelerometer and an intensity signal from the oximeter. From those signals the analyzer may determine parameters such as heart rate, heart rate variability, respiratory rate, arterial hemoglobin oxygen saturation, and exercise cadence. There may also be one or more processors electrically coupled to the signal analyzer, and a telemetry unit electrically coupled to the one or more processors. Further, the system may include a storage device having stored computer-executable instructions which, when executed by the one or more processors, implement a method. That method may include, receiving parameters from the signal analyzer, transforming the parameters from a time domain into a frequency domain, determining whether entrainment occurred, and determining whether lactic threshold has been reached by analyzing arterial hemoglobin oxygen saturation. Further, feedback may be transmitted to a remote receiver. The feedback may include a lactic threshold evaluation, and entrainment information to self-guide subjects in obtaining and perceiving a second wind.

A non limiting example of methods includes receiving parameters comprising heart rate, heart rate variability, respiratory rate, arterial hemoglobin oxygen saturation, and exercise cadence. The parameters may be transformed from the time domain into the frequency domain. The method may also include determining whether entrainment occurred, and whether lactic threshold has been reached by analyzing arterial hemoglobin oxygen saturation. Feedback may be transmitted to a remote receiver. The feedback may include a lactic threshold evaluation, and entrainment information to self-guide subjects in obtaining and perceiving a second wind.

A non limiting example relates to non-transitory computer readable medium having instructions stored thereon for execution by a processor. The computer readable medium includes instructions to receive parameters comprising heart rate, heart rate variability, respiratory rate, arterial hemoglobin oxygen saturation, and exercise cadence. Also, instructions to transform the parameters from a time domain into a frequency domain. The computer readable medium may also include instructions to determine whether entrainment occurred, and instructions to determine whether lactic threshold has been reached by analyzing arterial hemoglobin oxygen saturation. The instructions may further comprise instructions to transmit feedback to a remote receiver. The feedback may include a lactic threshold evaluation, and entrainment information to self-guide subjects in obtaining and perceiving a second wind.

One embodiment of a portable system for lactic threshold and entrainment detection may comprise a wearable pulse oximeter configured to measure heart rate and arterial hemoglobin oxygen saturation. The pulse oximeter may also produce a corresponding intensity signal. The system may further comprise an accelerometer configured to measure the motion of a user and produce a corresponding motion signal. Further, a signal analyzer may be electrically coupled to the pulse oximeter and the accelerometer. The signal analyzer may be configured to transform the intensity signal and the motion signal from a time domain into a frequency domain.

One or more processors may be used to analyze the signal. For example, the processors may detect heart rate variability in a transformed intensity signal, and exercise cadence in a transformed motion signal. Also, the processors may determine respiratory rate by examining the heart rate variability caused by respiratory sinus arrhythmia.

Further, the processors may compare respiratory rate, heart rate, and exercise cadence, for example, to determine whether entrainment has occurred. For example, entrainment may be determined to have occurred when respiratory rate, heart rate, and exercise cadence are all integer multiples of one another, without significant remainder. A telemetry unit electrically coupled to the one or more processors may be configured to transmit to a remote receiver feedback. For example, feedback may be entrainment information to self-guide a subject in obtaining and perceiving a second wind. For instance, entrainment information feedback may provide an indication of how close the subject is to obtaining entrainment.

Such a system may also determine whether lactic threshold has been reached by analyzing arterial hemoglobin oxygen saturation. In some embodiments wherein lactic threshold is determined to have been reached when the arterial hemoglobin oxygen saturation incurs an inflection point. For example, the inflection point may be incurred when there is at least a 1%-10%, preferably a 4% desaturation in arterial hemoglobin oxygen. The system may generate a lactic threshold evaluation based on the lactic threshold determination. This may be used to assist a user in determining how close the user is to the lactic threshold. For example, the feedback transmitted by the telemetry unit may include the lactic threshold evaluation.

Another embodiment may use an electrocardiogram belt with one or more of a signal analyzer, an accelerometer, at least two electrodes. The electrocardiogram belt may be used with the wearable pulse oximeter or by itself. The two electrodes may be electrically coupled to the signal analyzer and obtain a raw electrocardiogram high fidelity signal. In some embodiments, the signal analyzer may use a fast Fourier transform based spectral analysis to transform the electrocardiogram signal from the time domain to the frequency domain. Another embodiment may compare the transformed electrocardiogram signal with the transformed intensity signal to ensure the accuracy of the signals. The electrocardiogram belt may also include a telemetry unit electrically configured to wirelessly transmit signals obtained by the signal analyzer from the electrodes and accelerometer.

A non limiting example of a method includes receiving a heart rate signal, an arterial hemoglobin oxygen saturation value, and a motion signal. These values may be from a time domain into a frequency domain. For example, the heart rate signal may be a raw electrocardiogram high fidelity signal. In such an example, fast Fourier transform based spectral analysis may be used to transform the electrocardiogram signal from the time domain to the frequency domain. Heart rate variability may be detected in a transformed heart rate signal. Further, respiratory rate may be determined by examining the heart rate variability caused by respiratory sinus arrhythmia. The method may also detect exercise cadence in a transformed motion signal. And, the method may compare respiratory rate, heart rate, and exercise cadence to determine whether entrainment has occurred. For example, entrainment may be determined to have occurred when respiratory rate, heart rate, and exercise cadence are all integer multiples of one another, without significant remainder. Feedback comprising entrainment information to self-guide subjects in obtaining and perceiving a second wind may be transmitted to a remote receiver. For example, it may provide an indication of how close the subject is to obtaining entrainment.

In another embodiment, the method may also determine whether lactic threshold has been reached by analyzing arterial hemoglobin oxygen saturation. A lactic threshold evaluation may be generated and transmitted based on the lactic threshold determination. For example, lactic threshold evaluation may assess whether maximal lactate steady state has been reached, where maximal lactate steady state has been reached when lactate levels approach but do not crossing the lactic threshold. In some embodiments, the lactic threshold evaluation may provide instructions to guide a subject in obtaining and maintaining maximal lactate steady state. In some embodiments a determination of whether entrainment occurred and lactic threshold has been reached is done using the electrocardiogram signal and a pulse oximeter signal.

Another example of a method may provide assistance for high intensity interval training. The method may receive, from a pulse oximeter, an arterial hemoglobin oxygen saturation value. Further, it may determine, via a processor, whether a user has entered into the anaerobic training zone by analyzing arterial hemoglobin oxygen saturation. The time spent in the anaerobic training zone may be monitored, and after a designated time in spent training in the anaerobic training zone, instructions for a user to decrease training activity to enter in the aerobic training zone may be transmitted by a telemetry unit. It may also be determined whether a user has entered into the aerobic training zone by analyzing arterial hemoglobin oxygen saturation. The time spent in the aerobic training zone may be monitored, and after a designated time spent training in the aerobic training zone, instructions for a user to increase training activity to enter in the anaerobic training zone may be transmitted by a telemetry unit.

The designated time may be adjustable. In one embodiment the designated time spent in the anaerobic training zone and the designated time spent in the aerobic training zone may be altered by a user. In another example, the designated time spent in the anaerobic training zone and the designated time spent in the aerobic training zone are configured to automatically adjust according to a training program.

It should be understood that in the above described devices, apparatuses, systems and methods, and those elsewhere herein, that one or more of the features can be specifically excluded, and one or more other features described in connection with other devices, apparatuses, systems and methods, can be specifically added to or additional included.

### BRIEF DESCRIPTION OF THE DRAWINGS

Illustrative embodiments will hereafter be described with reference to the accompanying drawings.

FIG. 1 is a system diagram of a wearable lactic threshold and entrainment exercise device in accordance with an illustrative embodiment.

FIG. 2 is a cross sectional view of a wearable lactic threshold and entrainment exercise device in accordance with an illustrative embodiment.

FIG. 3 is a wearable lactic threshold and entrainment exercise device in accordance with an illustrative embodiment.

FIG. 4 is an electrocardiogram-based beat-to-beat heart rate variability monitoring device utilized as an optional subsystem of a wearable lactic threshold and entrainment exercise device in accordance with an illustrative embodiment.

FIG. 5 is a system diagram of an electrocardiogram-based beat-to-beat heart rate variability monitoring device utilized as an optional subsystem of a wearable lactic threshold and entrainment exercise device in accordance with an illustrative embodiment.

FIG. 6 is a flow chart illustrating an exemplary process for calculating lactic threshold and entrainment occurrence in accordance with an illustrative embodiment.

FIG. 7(a) and FIG. 7(b) are an ECG signal used to calculate lactic threshold and exercise entrainment in accordance with an illustrative embodiment.

FIG. 8 is a diagram of an FFT-based spectral analysis algorithm of the current LTEExD obtained from a simulated photo-plethysmogram (P(t)) under heavy exercise conditions, with resultant key frequency components and spectral lines (P(v)), including the cardiac peak and the motion peak in accordance with an illustrative embodiment.

FIG. 9 is a flow chart showing the major process steps taken by a remote display unit to show heart rate, HRV, respiratory rate, SpO2, exercise cadence, lactic threshold, and entrainment to an observer in accordance with an illustrative embodiment.

FIG. 10(a) and FIG. 10(b) illustrates two remote display units (smart-watch and smart-phone) in accordance with an illustrative embodiment.

### DETAILED DESCRIPTION

In the following detailed description, reference is made to the accompanying drawings, which form part of the present disclosure. The embodiments described in the drawings and description are intended to be exemplary and not limiting. As used herein, the term “exemplary” means “serving as an example or illustration” and should not necessarily be construed as preferred or advantageous over other embodiments. Other embodiments may be utilized and modifications may be made without departing from the spirit or the scope of the subject matter presented herein. Aspects of the disclosure, as described and illustrated herein, can be arranged, combined, and designed in a variety of different configurations, all of which are explicitly contemplated and form part of this disclosure.

Described herein are illustrative embodiments for methods and systems for a wearable lactic threshold and entrainment exercise device (LTEExD). In representative embodiments, an LTEExD non-invasively and directly measures Arterial Hemoglobin Oxygen Saturation (SpO2), Heart Rate (HR), Heart Rate Variability (HRV), Respiratory Rate (RR), and Exercise Cadence (RPM). These parameters, when taken together, allow the indirect derivation of blood pH and lactate levels. These parameters taken together provide important and key information regarding Lactic Threshold (LT), which can thus be effectively used in the prescription of heavy exercise. In addition, these same parameters may be utilized to provide important and key information regarding cardio-respiro-loco-motor synchronization (CRLS) and entrainment, which can thus be effectively used as a tool to improve the efficiency of heavy exercise, that is, point the observer towards gaining a ‘Second Wind’, through visual feedback on a remote display.

Entrainment, as is known to those skilled in the art, is the process whereby heart, lungs, and exercise cadence (that is, several interacting periodic systems) all become synchronized together, in order to become a more efficient machine. Coleman 1921, O'Rourke 1992, Schafer 1998, Rabler 1996, Niizeki 1993, Bechbache 1977, and Phillips 2013 have all described integer-based, factor-based, and phased-based coupling between the cardiovascular and respiratory and loco-motor systems. This coupling, which significantly improves exercise efficiency and performance, is the fundamental basis for entrainment. Exemplary LTEExD embodiments described herein may detect and alert a user of entrainment to significantly improve endurance and speed at the same work and power output.

Further, SpO2, HR, HRV, RR, and RPM variables are well known and are common independent measures in clinical, hospital, and critical care settings, and during exercise. However, measuring these variables in the field while exercising is much more difficult and challenging than measuring them in clinical settings. In fact, accurate, reliable, and reproducible measurement of the entire variable group has not yet been accomplished in real-time during outdoor exercise in the field. The LTEExD embodiments described herein utilizes three important principles in order to make the device work in the field under exercise conditions: (i) portable and wearable; (ii) digital signal processing and spectral analysis; and, (iii) wireless telemetry to a remote display. This combination of features is considered essential by coaches, trainers, experts, and athletes themselves in order to optimize the prescription of heavy exercise and improve competitive ability. For example, SpO2 is most easily measured by healthcare providers from the fingertip location, but this anatomical area is considered off limits for measurement purposes during exercise. In addition, outdoor lighting conditions and motion artifact increase the degree of difficulty for SpO2 measurement as well, and pulse oximeters made for clinical use are known to those skilled in the art to perform poorly under outdoor exercise circumstances.

Two key concepts for Lactic Threshold (LT) measurement are that core body temperature (Tcore) rises, and blood pH decreases, sometimes dramatically, when lactic threshold is reached, and exercise proceeds beyond LT into the anaerobic realm. For example, Tcore may change from 37° C. (normal) towards 41° C. (hyperthermia), and blood pH may drop from 7.40 (normal) towards 7.10 (acidemia), when LT is reached. As these changes occur, individual muscles, the cardiovascular system, and the central nervous system may become markedly less efficient, especially if LT is not practiced. Eventually as heavy exercise progresses, LT and anaerobic exercise may be reached, SpO2 typically incurs an inflection point at LT, and may drop from 98% (normal) towards 92% (desaturation) in a phenomenon labeled as exercise-induced hypoxemia (EIH). In one embodiment, LT is determined by finding that a 5% or more desaturation occurs.

LTEExD embodiments described herein detect SpO2 desaturation in the exercising athlete, in the field, by fast fourier transform (FFT) based spectral analysis, indirectly estimating decreasing blood pH and increasing blood lactate levels, thus accurately detecting the presence of Aerobic Exercise, Lactic Threshold, and Anaerobic Exercise, in real-time. Non-invasive lactic threshold monitoring by the LTEExD makes prescription of heavy exercise by trainers and coaches more precise and productive. For example, an LTEExD may effectively be utilized for the prescription of high intensity interval training (HIIT) and Tabata Intervals as described by Tabata 1996.

It is now clear through decades of research that LT is a critical exercise transition period representing the level of physical performance at which muscles just begin to produce more lactic acid than can be removed by liver and muscle enzyme systems. As LT is reached, maximal exertion becomes limited. Continued exertion above LT can last for only a few more minutes of anaerobic exercise as oxygen debt, hyperthermia, and lactic acid build up.

The lactate threshold (LT) is a useful measure for deciding exercise intensity for training and racing. LT varies between individuals and can be increased with training. High intensity interval training (H.I.I.T) takes advantage of the body being able to temporarily exceed the lactate threshold, and then recover (reduce blood-lactate) while operating below the threshold and while still doing physical activity. Fartlek and interval training are similar, the main difference being the structure of the exercise. Interval training can take the form of many different types of exercise and should closely replicate the movements found in the sport.

Within competition, LT has assumed a central role in real-time decision making for athletes and coaches ranging from Olympians to ‘age-groupers’ in triathlons. Precisely knowing the LT limit can be a key influence to guiding intra-race decisions, say to either accelerate and ‘go with’ a competitor who passes on the right, or simply ‘stay put and hold tight’ at the current race pace.

However, the ability to measure LT and provide this parameter to athletes is currently only available through the service of indoor, stationary, exercise physiology laboratories. For example, the gold standard for determining LT involves repeated sampling of blood via finger or ear needle pricks for blood lactate analysis. This procedure is time consuming, expensive, and uncomfortable, and especially inconvenient in the exercising athlete. Other prior art non-invasive LT testing methods are equally inconvenient and require additional and unwieldy apparatus worn on the face and head for measurement of respiratory gas exchange.

In contrast, the exemplary embodiments described herein non-invasively makes LT available to athletes, in the field, in real-time, during their work-out period through SpO2 monitoring. It is well known to those skilled in the art that at the point of LT, lactate increases from the anaerobic metabolism of glucose. Lactate is buffered in the blood, pH begins to decrease, and SpO2 begins to simultaneously decline. The increased acidity and body temperature resulting from LT causes SpO2 desaturation because the bonding force between O2 and Hb is weakened, as is well known to those in the art, through the science of the oxyhemoglobin dissociation curve.

In one embodiment, the specified LTEExD monitors primarily for SpO2 desaturation events through FFT-based spectral analysis, and thus provides for a portable, easy, and inexpensive way to detect LT in the field by athletes and coaches alike, and provides instantaneous feedback via remote display of the results. In an alternate embodiment, LT is detected through FFT-based spectral analysis of heart rate variability from a forehead photo-plethysmogram or electrocardiogram-based heart rate chest belt.

The described LTEExD may be used in real-time exercise in the field, and is especially useful for evaluating training adaptations. Coaches and athletes can easily monitor for entrainment, SpO2 desaturations, and heart rate variability changes, (through FFT-based spectral analysis by the signal analyzer and remote display of the results). In fact, coaches and athletes can identify important inflection points indicating LT at particular heart rate/respiratory rate/exercise cadence/speed/power combinations. Particular variable combinations may then be useful to better define and refine training zones and control training intensity, in preparation for a major competition event.

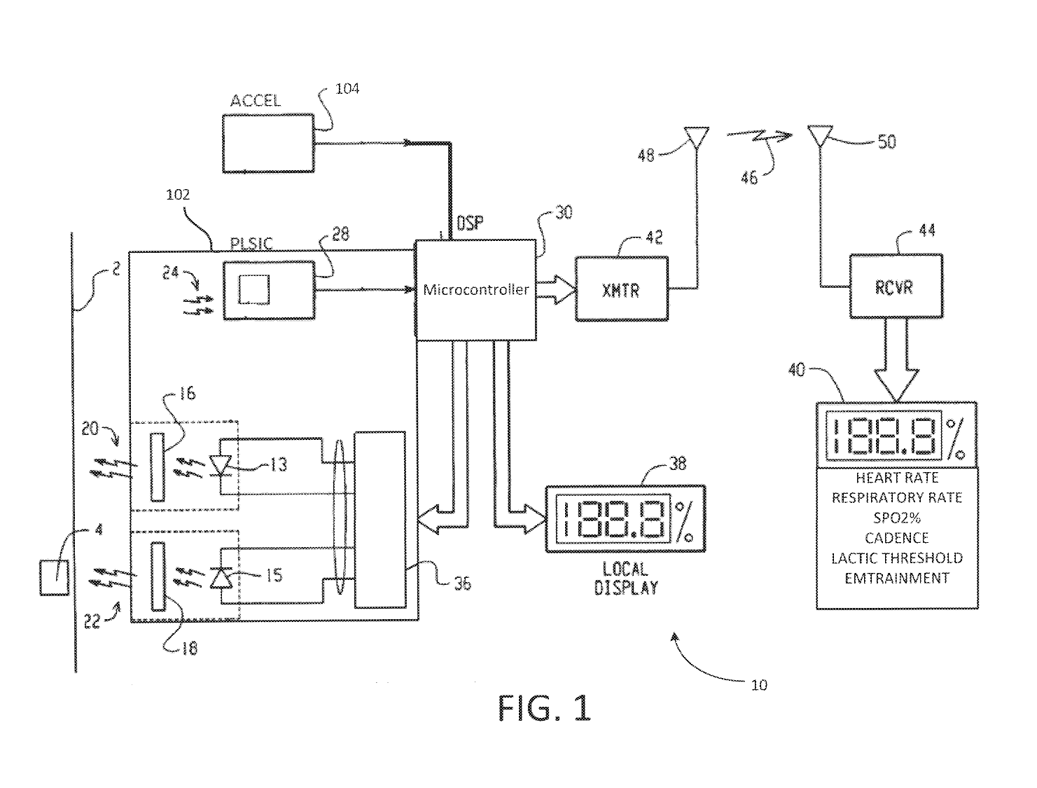

FIG. 1 illustrates a system diagram of an LTEExD 10 according to one embodiment. As shown, the LTEExD 10 may comprise a proximity-light-sensor integrated circuit (PLSIC) 102 and an Absolute Orientation Sensor (AO) 104 to determine heart rate, HRV, respiratory rate, SpO2, exercise cadence, lactic threshold, and entrainment. The LTEExD 10 may further comprise a microcontroller 30 to process the data from the Absolute Orientation Sensor 104, and a transmitter 42 and antenna 48 to send the data to a remote display 40. In other embodiments, a local display 38 may also be used to convey information to the user.

In one embodiment, the Absolute Orientation Sensor 104 may include 3-axes directions of detectable accelerations, digital sequencer and control logic, in a single chip configuration. The Absolute Orientation Sensor 104 may be placed with the PLSIC 102, or placed on or near the human body's center of gravity, such as forehead, chest, or against the posterior midline skin on the lumbar or thoracic spine. In another alternative embodiment, RPM is obtained from an Absolute Orientation Sensor sub-system held close to the skin of the body at a center of gravity location, including: small of the back; waistline belt at posterior lumbar spine; pocket of pants at posterior lumbar spine; or, pocket of shirt or jersey at posterior thoracic or lumbar spine.

In certain embodiments, the Absolute Orientation Sensor 104 may facilitate RPM analysis. In one embodiment, the Absolute Orientation Sensor 104 may output a serial digital pulse train corresponding to the physical motion of the exercisers body. For example, a periodic electrical signal generated by the Absolute Orientation Sensor 104 may have a value associated with the motion in the x-, y-, and z-axis. In such embodiments, a signal analyzer (e.g., microcontroller 30) may complete the square root of the sum of the squares of the axes. This may provide a value in linear relationship to the value associated with the intensity of the periodic exercising motion. Thus, a data stream may be generated corresponding to the physical motion of the exercising subject.

In some embodiments, the PLSIC 102 may include a single power supply, LED drivers 36, photodiodes 28, digital sequencer and control logic, in a single chip configuration. This allows the LTEExD 10 device to be smaller and have fewer parts than previous devices.

For example, a typical prior art oximeter has a photodiode for detecting an optical signal reflected from or transmitted through a volume of intravascular blood illuminated by one or more light emitting diodes. The LEDs emit electromagnetic radiation at a constant intensity; however, an optical signal with a time-varying intensity is transmitted through or reflected back from the intravascular blood for each of the wavelengths. The photodiode generates a low-level current proportional to the intensity of the electromagnetic radiation received by the photodiode. The current is converted to a voltage by a current to voltage converter, which may be an operational amplifier in a current to voltage (transimpedance) configuration. The signal is then filtered with a filter stage to remove unwanted frequency components, such as any 60 Hz or 120 Hz noise generated by incandescent and fluorescent lighting. The filtered signal is then amplified with an amplifier and the amplified signal is sampled and held by a sample and hold while the signal is digitized with a high-resolution (12-bit or higher) analog to digital converter (ADC). The digitized signal is then latched by the central processing unit (CPU) of the computer system from the ADC. The computer system then calculates a coefficient for the oxygen saturation value from the digitized signal and determines the final saturation value (SpO2) by reading the saturation value for the calculated coefficient from a look-up table stored in memory. The final saturation value and heart rate are displayed on an integrated display directly connected to the CPU.

Thus, the generic prior art pulse oximeter requires numerous devices to determine the oxygen saturation value from the optical signal. Moreover, these devices, particularly the ADC, require a relatively large amount of space and electrical power, thereby rendering a portable unit impractical.

As illustrated, in some embodiments of the LTEExD 10, the photodiode, current to voltage converter, filter, amplifier, sample and hold, and analog-to-voltage converter are replaced with the PLSIC 102 and an Absolute Orientation Sensor 104. Replacing all of the components in the prior art pulse oximeter reduces the footprint, power consumption, and parts count of embodiments of the LTEExD over prior art systems.

The PLSIC 102 may be used for detecting a multiplexed optical signal 24 from a volume of intravascular volume of blood 4, under the skin 2, and illuminated by two (or more) wavelengths of light emitting diodes (LEDs) 13 and 15, with lenses 16 and 18. The LEDs emit electromagnetic radiation at a constant intensity; however, an optical signal 24 with a time-varying intensity is transmitted through or reflected back by the intravascular blood for each of the wavelengths. In a preferred embodiment, the multiplexed and reflected optical signal 24 is analyzed to determine the arterial hemoglobin oxygen saturation value (SpO2). For example, in some embodiments, the PLSIC 102 produces a periodic electrical signal in the form of a digital serial pulse train corresponding to the intensity of the broadband optical signal transmitted through or reflected by the intravascular blood under the skin.

In some embodiments a first LED 13 is a red LED, emitting light having a wavelength of approximately 660 nm. In other embodiments LED 13 may emit a wavelength of light in the 600-750 nm spectrum. However, because a solution of human hemoglobin has an absorption maximum at a wavelength of about 660 nanometers (red), the closer to that wavelength, the more accurate the results (otherwise, various new calibration curves are required for SpO2 calculation, as is known in the art).

Further, in one embodiment, a second LED 15 is an infrared LED, emitting electromagnetic radiation having a wavelength of approximately 805 nm. In other embodiments LED 15 emits a wavelength of light between the 805-940 nm. However, the 805 nm isobestic point of hemoglobin absorption is particularly useful and suitable for the ir-LED 15. At this point the absorption of the emitted electromagnetic radiation by the blood 4 is unaffected by the presence or absence of oxygen bound to the hemoglobin molecules.

The LED drivers 36 may be any driver capable of providing a signal capable of causing one or more LEDs to illuminate. In some embodiments, an LED-driving circuit may have integrated LED drivers 36 which allow the LEDs 13 and 15 to be alternatively illuminated (i.e. multiplexed) under control of the microcontroller 30. In another embodiments, these LED drivers 36 may have a normalizing function (e.g. for different hues of skin and volumes of blood) that increases or decreases the intensity of electromagnetic radiation generated by the LEDs in the system, as needed. In such embodiments, the ability of the microcontroller 30 to automatically vary the LED intensity via the LED drivers 36, and the photodiode 28 sensitivity by the photodiode drivers, provides for a sensitive optical system with very wide dynamic range, including capability for outside daylight use in the field.

The PLSIC's 102 LED drivers 36, LEDs 13 and 15, and photodiodes 28 may be used to detect SpO2. The degree of SpO2 desaturation is a vital index of the condition of an exerciser. As blood is pulsed through the lungs by the heart action, a certain percentage of deoxy-hemoglobin (RHb) picks up oxygen so as to become oxy-hemoglobin (HbO2). From the lungs, the blood passes through the arterial system until it reaches the capillaries at which point a portion of the HbO2 gives up its oxygen to support the life processes in adjacent cells. As a unique physiologic adaptation of the human body during heavy exercise, relatively more oxygen is released by the HbO2 complex (desaturation) at LT when blood pH is significantly decreased and Tcore is significantly increased.

By definition, SpO2=HbO2/(RHb+HbO2). A healthy, conscious person will have an oxygen saturation of approximately 96 to 98%. For example, during clinical use, the primary use of pulse oximetry is to detect and prevent scenarios when a person loses consciousness or suffers permanent brain damage if SpO2 falls to very low levels for extended periods of time. Conversely, embodiments of the LTEExD as described herein are designed for exercise use in the field, and still determines SpO2 by analyzing the change in color of the blood.

When radiant energy passes through a liquid, certain wavelengths may be selectively absorbed by particles which are dissolved therein. For a given path length that the light traverses through the liquid, the Beer-Lambert relation indicates that the relative reduction in radiation power (P/Po) at a given wavelength is an inverse logarithmic function of the concentration of the solute in the liquid that absorbs that wavelength. For a solution of oxygenated human hemoglobin, the absorption maximum is at a wavelength of about 660 nanometers (red). Therefore, instruments that measure absorption at this wavelength are capable of delivering useful information as to oxy-hemoglobin levels.

It is well known to those skilled in the art that RHb absorbs more red (660 nm) light than HbO2, and that absorption of infrared (805 nm) electromagnetic radiation is relatively unaffected by the presence of oxygen in the hemoglobin molecules. Thus, some embodiments of the LTEExD may determine SpO2 by: (1) alternatively illuminating a volume of intravascular blood with electromagnetic radiation of two selected wavelengths (red and infrared wavelengths); (2) converting the time-varying electromagnetic radiation intensity transmitted through or reflected back by the intravascular blood for each of the wavelengths from the time domain into the frequency domain by FFT-based spectral analysis; and, (3) calculating SpO2 values for the exercisers blood by applying the Lambert-Beers transmittance law to the detected transmitted or reflected electromagnetic radiation intensities at the selected wavelengths, that is, by analyzing, comparing, and dividing the AC/DC values of each wavelength (R-value), and empirically correlating the resultant R value to SpO2 by equation or look-up table.

The microcontroller 30 may be any computer system capable of performing variable calculations and digital signal processing to the desired accuracy in the desired period of time. The microcontroller 30 interfaces with the PLSIC 102, Absolute Orientation Sensor 104, a local and remote display 38 and 40, LEDs 13 and 15, LED drivers 36, transmitter 42, and wireless telemeter system 46 48 50.

In some embodiments, the microcontroller 30 may include a CPU, random access memory (RAM), and read-only flash memory (ROM). Further, the microcontroller 30 may be capable of being a signal analyzer. That is, the microcontroller 30 may have the computational capacity for digital signal processing from the time domain into the frequency domain to determine the heart rate, HRV, respiratory rate, oxygen saturation, lactic threshold, and entrainment values from the periodic serial digital pulse streams from the PLSIC, Absolute Orientation Sensor, and electrocardiogram chest belt HRV data streams.

For instance, once inside the microcontroller 30 with hardware floating point unit of the present LTEExD, the PLSIC 102, Absolute Orientation Sensor 104, and electrocardiogram chest-belt sensor signals are analyzed to determine the heart rate, HRV, respiratory rate, oxygen saturation, exercise cadence, lactic threshold, and entrainment values. In one embodiment, PLSIC light intensity data, Absolute Orientation Sensor acceleration (meters/second/second or g's) data, and chest belt electrocardiogram beat-to-beat HRV data, are all converted into the frequency domain by performing the well-known Fast Fourier Transform (FFT) on the data by the microcontroller 30. In other embodiments, other common techniques of converting time-domain data to the frequency domain may be used: e.g., discrete cosine transform, wavelet transform, discrete Hartley transform, Gabor transform, Auto-regressive (AR) Spectral Estimation, and the Lomb-Scargle (LS) periodogram.

The frequency domain data may then analyzed to determine the heart rate, HRV, respiratory rate, oxygen saturation, exercise cadence, lactic threshold, and entrainment values. A signal analyzer may be used to compare and integrate the PLSIC, Absolute Orientation Sensor, and electrocardiogram chest belt HRV digital data streams by carrying out the calculations and analysis in firmware or software code executing on the microcontroller 30, smartwatch and/or smartphone.

In other embodiments, a suitable computer system for digital signal processing includes both a smartphone and smartwatch. For example, microcontroller 30 may perform FFT-based spectral analysis on the variables, optional data storage and post-processing calculations are then completed by a smartphone or smartwatch. Suitable smartwatch and smartphone remote displays 40 are capable of data storage and additional digital signal processing.

In an alternate embodiment, a smartphone, smartwatch, or computer system may perform the same signal analysis as the microcontroller 30, including conversion of the PLSIC, Absolute Orientation Sensor, and electrocardiogram chest belt HRV data streams from the time domain to the frequency domain.

In addition to performing heart rate, HRV, respiratory rate, oxygen saturation, exercise cadence, lactic threshold, and entrainment values, the microcontroller system 30 controls LED drivers 36, which controls the red-LED 13 and infrared-LED 15, and acquires the serial data streams from PLSIC 102, Absolute Orientation Sensor 104, and an optional electrocardiogram chest belt HRV. The microcontroller 30 may send the data stream to the transmitter 42.

As shown, the transmitter 42 may and telemeter 46 the data wirelessly to a remote display 40. The transmitter 42, receiver 44, and the two antennas 48 and 50, may be any suitable radio frequency or other wireless telemetry system. These telemetry systems are well known in the art and widely available. Bluetooth and WiFi telemetry protocols may be used to allow highly secure and noise-immune telemetry of desired values and variables even in noisy exercise environments. In one embodiment, multi-protocol bluetooth 4.0 low energy, 2.4 GHz, radiofrequency (RF), system-on-a-chip (SoC), technology provides a highly secure link, high noise immunity, and a high informational capacity. These telemetry traits are highly desirable in the wearable exercise device environment. In an alternative embodiment, transmitter 42 may transmit on both or either Bluetooth 4.0 and Bluetooth Classic data streams to both or either smartphones and smartwatches, as well as to any computer with a Bluetooth connection.

Once the values are calculated and telemetered 46, the values may be displayed to an observer (including self) on a local 38 or remote display 40. In some embodiments, the remote display 40 may be a smartphone or smartwatch.

The local and remote displays 38, 40 may display information about the detected and analyzed data. For example, the remote display 40 may be any display capable of displaying one or more heart rate, HRV, respiratory rate, oxygen saturation, exercise cadence, lactic threshold and entrainment values to the desired resolution. Liquid crystal displays (LCDs) are well known in the art, and ideal for certain embodiments of the LTEExD. In addition, a stack of discrete LEDs, or tricolor LED(s), may be used if the designer desires to display binary, tertiary, or logarithmic variable values. In an embodiment, green, yellow, and red discrete LEDs, or a tricolor LED, or color stripes on an LCD, may be configured to represent baseline, approaching, and desired conditions corresponding to lactic threshold and entrainment exercise.

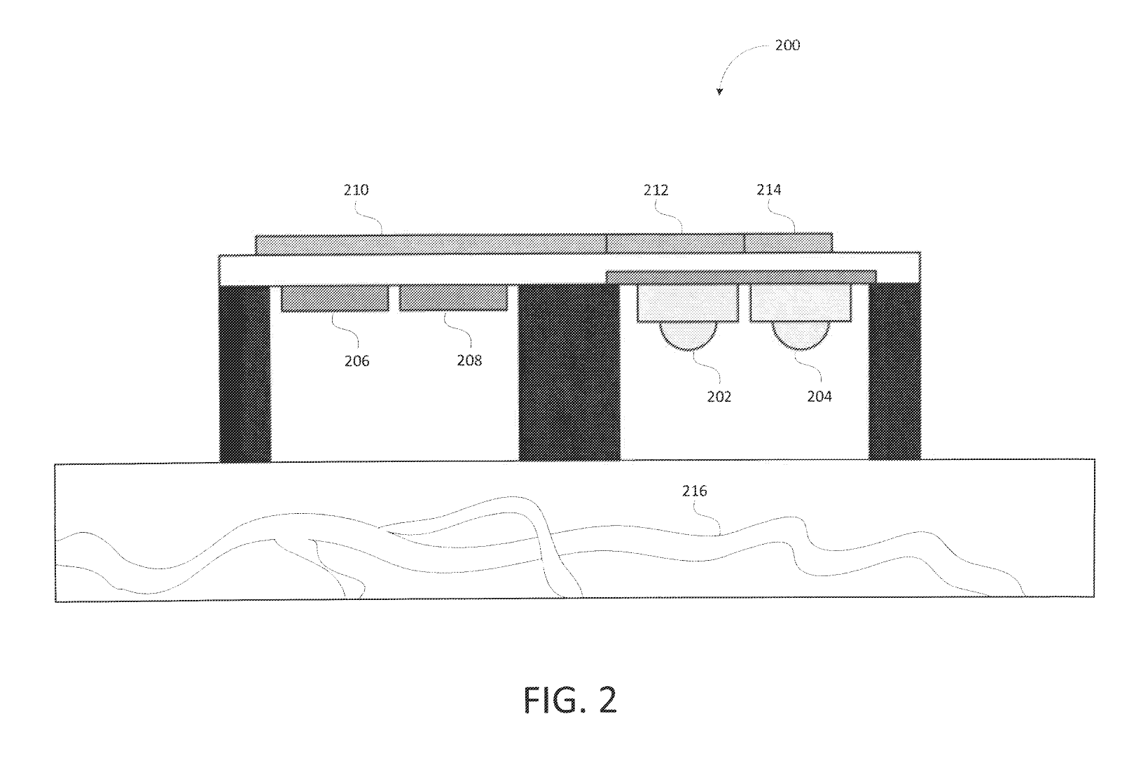

FIG. 2 is a cross sectional view of an LTEExD 200 in accordance with an illustrative embodiment. One embodiment may use a pair of light emitting diodes (LED 202, 204), a proximity-light-sensor integrated circuit (PLSIC 206), an Absolute Orientation Sensor 208, an optional electrocardiogram chest belt, a microcontroller (210) with hardware floating point unit, a signal analyzer (212) with FFT-based spectral analysis, a telemetry unit (214), and a remote wireless display unit.

According to one embodiment of the LTEExD 200, two light emitting diodes (LEDs 202, 204), a red LED 202 and an infrared LED 204, alternatively illuminate an intravascular blood 216 sample with two wavelengths of electromagnetic radiation. The electromagnetic radiation interacts with the blood 216 and a residual optical signal is both reflected and transmitted by the blood. A photodiode in the PLSIC 206 collects oximetry data from the intravascular blood 216 sample illuminated by the two LEDs 202, 204. The PLSIC 206 may produce a serial digital pulse train, the logarithm of which is proportional to the intensity of the optical signal. If the PCB has IR transmittance a ground fill may be used to prevent leakage.

In addition, an optional electrocardiogram chest belt may produce a serial digital pulse train of beat-to-beat HRV intervals. The serial digital pulsatile signals are then in a form suitable to be entered into the microcontroller 210 and signal analyzer 212.

Once inside the microcontroller 210, the time-domain data may be converted into the frequency domain by the hardware floating point unit via FFT-based spectral analysis. In some embodiments, the frequency domain data may be processed to determine SpO2, heart rate, HRV, respiratory rate, exercise cadence rate, lactic threshold, and entrainment.

Some embodiments may provide a portable, low-power, wireless, real-time device, with signal analyzer 212 using FFT-based spectral analysis, which analyzes lactic threshold and entrainment in the field, under heavy exercise, including training and competition conditions, and telemeters the variable results to an observer with remote display. The signal analyzer 212, according to one embodiment, may be a floating point unit (FPU) integrated with a microcontroller 210. The signal analyzer 212 may utilize fast fourier transform (FFT)-based spectral analysis to transfer the data from the time domain to the frequency domain.

The FPU-microcontroller 210 may interface to a telemetry unit 214. The calculated HR, HRV, RR, SpO2, RPM, lactic threshold, and cardio-respiro-locomotor synchronization (entrainment) variables may be telemetered to a remote display located on an observer (self, trainer, coach, and/or fan) via the telemetry unit. The remote display may be used to self-guide subjects in improving athletic performance in the field, and obtain and perceive their second wind, through visual feedback on a remote display.

As shown, one embodiment of the LTEExD 200 use of a PLSIC 206, Absolute Orientation Sensor 208, and Bluetooth 4.0 SoC (telemetry unit 214), with optional electrocardiogram chest belt, allows for a truly portable heart rate, HRV, respiratory rate, exercise cadence rate, arterial hemoglobin oxygen saturation, lactic threshold, and entrainment device. In addition, microcontroller 210 with hardware floating point unit and digital signal processor may be available in ball grid array (BGA) form (the monolithic electronic device without external packaging or leads), allowing a multi-chip module (MCM) to be fabricated by connecting the sensors and components at the SoC-level. Further, in some embodiments LED drivers may already integrated into the PLSIC 206. Thus, an extremely small device can be constructed for real-time use in the field under daylight and exercise conditions.

According to some embodiments, the specified LTEExD 200 is light and small enough to be worn during heavy exercise. That is, the device can be made light enough and otherwise configured to be worn by an exerciser in the manner that a wrist watch, bracelet, chest belt, anklet, inflatable cuff, Velcro band, elastic band, sweatband, headband 300 (as shown in FIG. 32), cap, hat, or helmet might be worn.

For example, in one embodiment, LEDs 202 and 204, PLSIC 206, microcontroller 210, and a remote display may be packaged in an inflatable cuff system with the LEDs 202 and 204 and PLSIC 206 placed in optical communication with the skin. Further, a local display may be positioned to be readable by self, coach, or trainer.

As will be understood by one skilled in the art, virtually any wearable exercise device design could be modified to use the PLSIC 206 and Absolute Orientation Sensor 208 for lactic threshold and entrainment detection. Therefore, the embodiments are not limited to the specific details, representative apparatus and method, and illustrative examples shown and described. Accordingly, departures may be made from such details without departing from the spirit or scope of the applicant's general inventive concept.

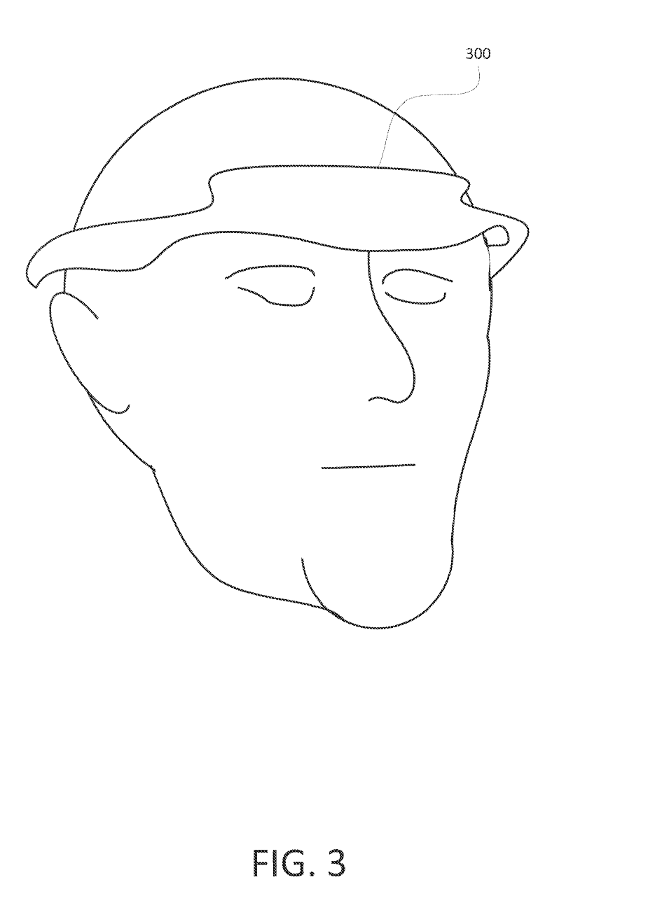

FIG. 3 is a wearable LTEExD 300 in accordance with an illustrative embodiment. As shown, the LTEExD 300 may be located on the forehead of a user. An embodiment located on the forehead may use pulse oximetry on the supra-orbital artery perforating the skull at eyebrow location, originating from the internal carotid artery.

In one embodiment the LTEExD 300 may comprise the two LEDs, a PLSIC, an Absolute Orientation Sensor, a microcontroller, and a Bluetooth 4.0 transmitter packaged in a single printed circuit board with a single power supply. The single printed circuit board may be packaged into a small printed circuit board about the size and configuration of a headband. The LEDs and PLSIC may be placed in optical communication with the skin and the intravascular blood beneath. An antenna may also be positioned within the headband, which can wrap around or otherwise encircle the head anatomy to secure the package during heavy exercise. In an alternative embodiment, the antenna may be positioned externally of the package.

The size and positioning of the LTEExD 300 may allow for it to be used during various activities. Thus, whether running on the track, cycling on the road, or swimming in the pool, athletes can see what is happening during heavy exercise on a remote display, and potentially manipulate their exercise intensity as they approach and breakthrough LT. For example, LT may be approached but not crossed over in a time-trial competitive race event in order to utilize energy most efficiently. On the other hand, LT may be broken through programmatically during interval training for maximal training benefit. With this information in hand, athletes, trainers and coaches can measure and improve work output more precisely, and thus improve overall training regimens and competitive ability considerably.

Prior art pulse oximeters have a large desktop footprint because of the circuitry heretofore believed necessary to capture the signals. Such higher-powered circuitry shortens battery life. Typical pulse oximeters use a silicon photodiode, a current-to-voltage converter (a transimpedance amplifier), a preamplifier, filter stage, a sample and hold, and an analog-to-digital (ND) converter to capture the oximetry signal. These components make the creation of truly portable oximeters for use in the field difficult because of the large footprint and high power requirements of each device. The A/D converter, in particular, is typically large and power-hungry.

Importantly, embodiments of the LTEExD 300 are able to resolve all these size and power issues through a minimal microchip design philosophy: one-chip photodiode with LED drivers, with serial data stream output; one-chip microcontroller, with digital signal processor, floating-point-unit, and signal analyzer; one-chip telemeter, with low power Bluetooth protocols; two LEDs; and, a small single supply battery sub-system. In this manner, the LTEExD 300 may efficiently and portably calculate the desired lactic threshold and entrainment variables in the field under heavy exercise conditions.

Further, signal artifact from exercise motion, ambient light, and low perfusion (low blood circulation through the extremities) are primary causes of inaccurate and imprecise SpO2 readings. (“Artifact” is any component of a signal that is extraneous to the variable represented by the signal.) Inaccuracies are also caused from physiologic nonlinearities and the heuristic methods used to arrive at the final saturation values. The LTEExD 300 again solves these classic exercise issues through the use of FFT-based spectral analysis to transfer the data from the time domain to the frequency domain, which provides much improved digital signal processing capability to calculate the variable values outdoors in the field setting, including in ambient light, during sunlight hours, during exercise motion, and during low perfusion scenarios.

SpO2 signal artifact has three major sources: (1) ambient light (which causes DC signal offset, and inaccurate R values); (2) low perfusion (which causes the intensity of the desired AC signal to be very low, and inaccurate R values); and (3) exerciser or sensor motion (which generates a large AC/DC artifact, masking the desired signal, and inaccurate R values). When the oximetry signal is amplified, the noise components are amplified along with the desired signal. This noise acts to corrupt the primary signal, during both pre-processing as well as post-processing, thereby reducing the accuracy of the SpO2 reading. Signal artifact is prevalent with both reflectance- and transmittance-type probes. The LTEExD 300 resolves all three of these signal artifact issues through hardware, firmware, and software design, and, as a result, is fully able to calculate and resolve the desired lactic threshold and entrainment variables in the field.

For example, the forehead location of the LTEExD 300 may resolve some artifact issues. Yamaya 2002 described the use of pulse oximeters during heavy exercise in an indoor exercise lab environment. Although SpO2 finger sensors are well-accepted for use in resting subjects, their accuracy and use during heavy exercise has always been problematic in prior art designs due to motion artifact and low perfusion. SpO2 finger sensors in particular used during heavy exercise are subjected to varying degrees of motion, often resulting in signal corruption. Also, certain types of exercise like cycling often result in weakening or total loss of the finger SpO2 waveform, due to low perfusion, because of gripping the handlebars or another object. In addition, diversion of large amounts of blood to the working muscles during heavy exercise makes accurate SpO2 detection difficult as well, again due to low perfusion. Although not tested for in Yamaya's indoor exercise study, outdoor sunlight in the field may also easily oversaturate the photodiode and thus corrupt the SpO2 signal.

On the basis of improving SpO2 accuracy during exercise in an indoor exercise controlled lab setting, Yamaya 2002 continued a series of demonstrations from the prior decade of an improved SpO2 sensor sub-unit through the use of the forehead location, secured by a headband. The method demonstrated here by Yamaya in 2002 notably utilized relatively simple digital signal processing through a Kalman Filtering technique. This experiment supported several other early investigations that also concluded that the forehead SpO2 sensor location offers major advantages by avoiding the severe inaccuracies seen with finger sensors due to gripping and motion, and also notably avoided the mechanical instability typically seen with ear lobe sensors.

In fact, it was shown by Yamaya and others that an easily secured compressive headband (or alternate method of utilizing pressure on the forehead probe) improves reflectance SpO2 waveform accuracy by preventing susceptibility to contamination from venous blood. Notably, the important concept of improved SpO2 accuracy at the forehead by pressure on the sensor probe site is also demonstrated by Dassel 1995, Cooke and Scharf 2004, Shelley 2005, and even in its earliest conceptions by Tammeling 1957, Mendelson 1988, and Takatani 1991. In addition, significant improvements in minimizing the delay time to detection of desaturation in stationary subjects was demonstrated by Cooke and Scharf 2002 through use of the forehead SpO2 sensor location. Minimizing delay time until detection of desaturation is noted to be due to the forehead blood supply originating from the supra-orbital artery, which is an end-artery of the internal carotid artery.

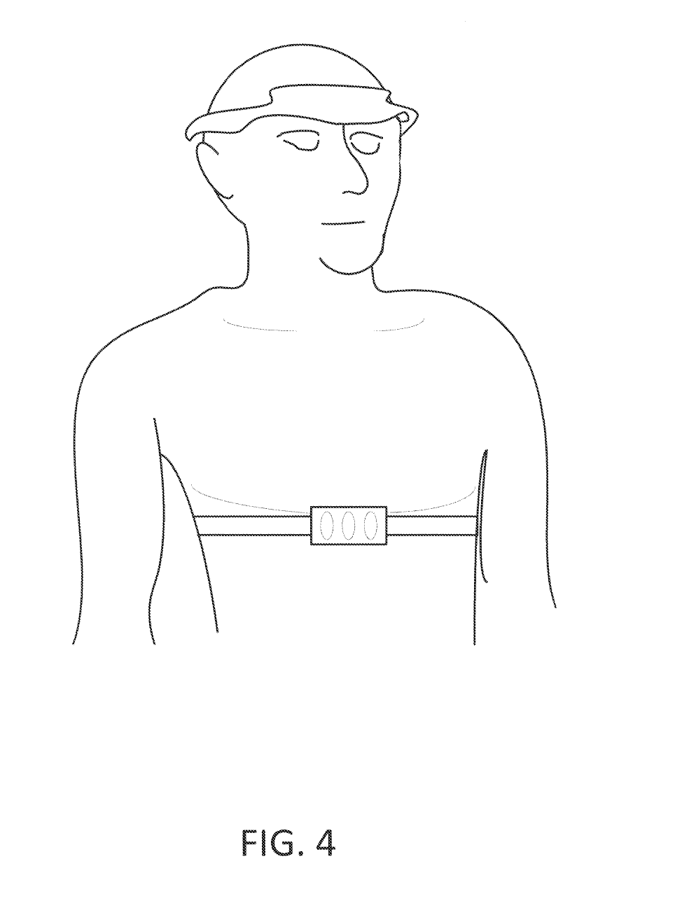

FIG. 4 is an electrocardiogram-based beat-to-beat heart rate variability monitoring (HRV) device utilized as an optional subsystem of an LTEExD 400 in accordance with an illustrative embodiment. As described above, an LTEExD 400 may include a portable pulse oximeter sub-system with a proximity-light-sensor integrated circuit (PLSIC) and Absolute Orientation Sensor as sensors. In addition, an optional electrocardiogram chest belt 402 and Absolute Orientation Sensor may also be used to obtain the HR, HRV, RR, and RPM variables.

The electrocardiogram chest belt 402 may deliver beat-to-beat HRV data to the signal analyzer within the LTEExD 400. In an alternative embodiment, an electrocardiogram chest belt 402 is integrated with an Absolute Orientation Sensor, and used to calculate HR, HRV, RR, RPM, lactic threshold and entrainment variables. In yet another embodiment, a signal analyzer in the chest belt calculates the HRV, and transmits the HRV data to the signal analyzers in the LTEExD 400, as well as a user device such as a smartwatch or smartphone.

HRV is the physiological phenomenon of variation in the time interval between heartbeats. It is measured by the variation in the beat-to-beat interval where R is a point corresponding to the peak of the QRS complex of the electrocardiogram (ECG) wave; and HRV is the interval between successive R waves. Methods known to those skilled in the art used to detect heart beats and their variation include: ECG, blood pressure, and the pulse wave signal derived from a pulse oximeter signal, known as the photo-plethysmogram (PPG). ECG is considered superior because it provides a clear, accurate, and precise QRS waveform, which makes it easier to analyze, and exclude heartbeats not originating in the sinoatrial node of the heart electrical system.

HRV is related to autonomic nervous system activity. The main inputs are from the sympathetic (SNS) nervous system, parasympathetic nervous system (PSNS), and humoral factors. Respiration gives rise to waves in heart rate mediated primarily via the PSNS, called respiratory sinus arrhythmia (RSA), and during exercise may be found in the 0.15-1.00 Hz range. SNS activity represents Traube-Hering-Mayer (THM) waves in the 0.03-0.15 Hz range. Factors that affect the THM and RSA waves are the baroreflex, thermoregulation, hormones, sleep-wake cycle, meals, physical activity, heavy exercise, and stress.

Both autonomic nervous system and respiratory system activity is present in physiologic waveforms, including both the photo-plethysmogram (PPG) and electrocardiogram (ECG). Regarding low frequency (LF) THM waves (0.03-0.15 Hz), decreased PSNS activity or increased SNS activity will result in reduced HRV. Regarding high frequency (HF) RSA waves (0.15 to 0.40 Hz, and up to 1.00 Hz during heavy exercise), these waves are a vagally mediated modulation of heart rate that increases during inspiration and decreases during expiration.

There are two primary LF-THM and HF-RSA fluctuations:

(1) Respiratory sinus arrhythmia (HF-RSA) causes a heart rate variation associated with respiration and faithfully tracks the respiratory rate across a range of frequencies (0.15-0.40 Hz, up to 1.00 Hz during heavy exercise).
(2) Low-frequency oscillations cause a heart rate variation associated with Traube-Hering-Mayer (LF-THM) waves in the range of 0.03-0.15 Hz, or about a 10-second period.

The most widely used methods to calculate HRV can be grouped under time-domain and frequency-domain analysis. All prior art wearable exercise devices all utilize a simple time domain method based upon the standard deviation of HRV. A simple time-domain formula is used that judges HRV on the basis of the geometric properties of the resulting pattern.

Contrastingly, embodiments of the LTEExD 400 convert ECG and PPG waveform data from the time-domain to the frequency domain using FFT-based spectral analysis. This frequency domain method, never before utilized in exercise devices in the field, assigns bands of frequency and then counts the intensity of HRV that matches each band. The HRV bands for analysis are typically high frequency (HF-RSA) from 0.15 to 1.0 Hz and low frequency (LF-THM) from 0.03 to 0.15 Hz.

For example, in certain embodiments, the LTEExD 400 transforms HRV data from the time domain to the frequency domain utilizing the fast fourier transform (FFT), and calculates power spectral density (PSD) for each frequency band. In an alternative embodiment, autoregressive (AR) spectral estimation is utilized. The FFT method is preferred and offers: (1) simplicity of the algorithm; and, (2) high processing speed. In the alternative embodiment, the advantages of AR are: (1) smoother spectral components that can be distinguished independent of preselected frequency bands; (2) easy post-processing of the spectrum with an automatic calculation of low- and high-frequency power components with an easy identification of the central frequency of each component; and, (3) an accurate estimation of PSD even on a small number of samples on which the signal is supposed to maintain stationarity.

Yet another alternative embodiment may use the Lomb-Scargle (LS) periodogram. LS can produce a more accurate estimate of the PSD than FFT methods for typical HRV data. Since HRV is an unevenly sampled data, the main advantage of the LS method is that, in contrast to FFT-based methods, LS is able to be used without the need to resample and detrend the HRV data.

For appropriate HRV accuracy and precision, certain embodiments may record of approximately one minute: the lowest bound of HF-RSA is 0.15 Hz (6.3 cycles/min); while the lowest bound of LF-THM component is 0.03 Hz (1.8 cycles/min).

Although cardiac automaticity is intrinsic to various pacemaker tissues, heart rate and rhythm are largely under the control of the autonomic nervous system. The parasympathetic influence on heart rate is mediated via release of acetylcholine by the vagus nerve. The sympathetic influence on heart rate is mediated by release of epinephrine and norepinephrine. Under resting conditions, vagal tone prevails and variations in heart period are largely dependent on vagal modulation. However, it is important to note that vagal and sympathetic activity constantly interact with each other.

HRV present during resting conditions represent beat-by-beat variations in cardiac autonomic inputs. Efferent vagal (parasympathetic) activity is a major contributor to the HF-RSA component. The LF-THM component is mainly a marker of sympathetic modulation, but may also represent both sympathetic and vagal influences. For example, during sympathetic activation the resulting tachycardia is usually accompanied by a marked reduction in total power, whereas the reverse occurs during vagal activation.

It is important to note that HRV measures fluctuations in autonomic inputs to the heart rather than the mean level of autonomic inputs. Thus, both withdrawal and saturatingly high levels of autonomic input to the heart can lead to diminished HRV.

Monitoring exercise training using an LTEExD 400 with electrocardiogram chest belt 402 may decrease cardiovascular mortality and sudden cardiac death. Regular exercise training is also thought to modify cardiac autonomic control. Individuals who exercise regularly have a ‘training bradycardia’ (i.e., low resting heart rate) and generally have higher HRV during rest periods than sedentary individuals.

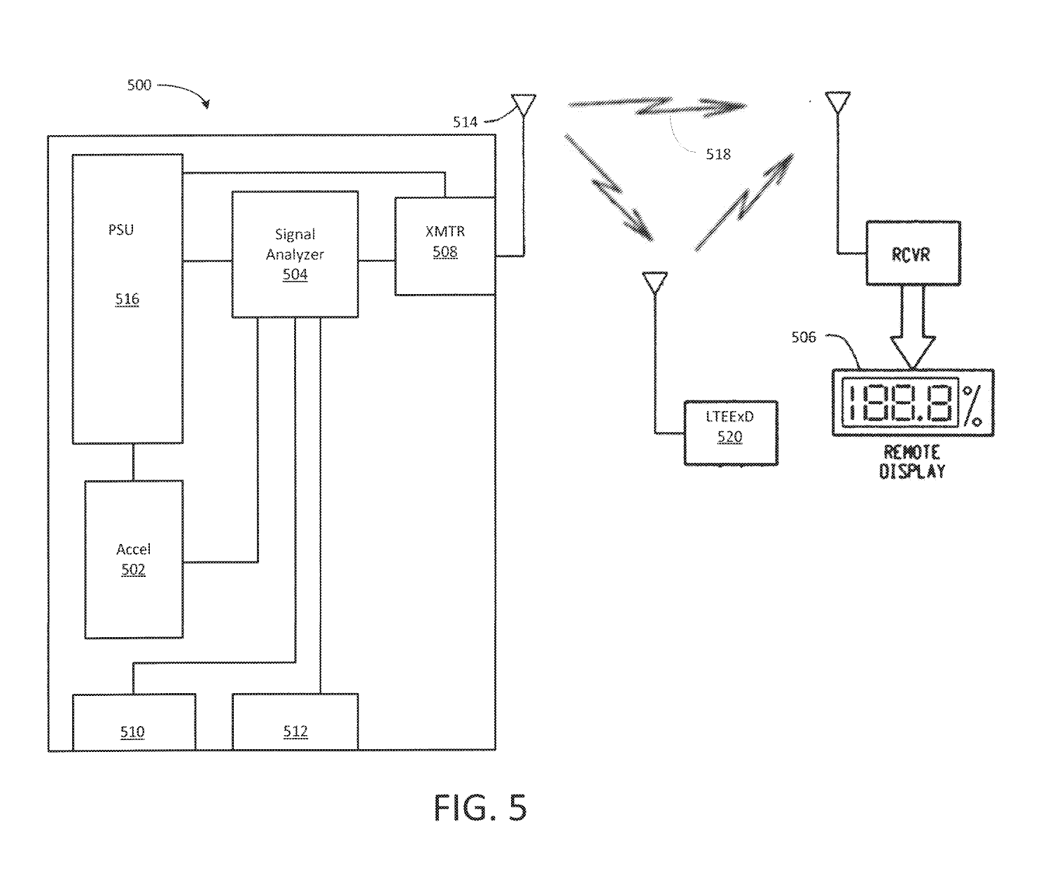

FIG. 5 is a system diagram of an electrocardiogram-based beat-to-beat HRV monitoring device 500 in accordance with an illustrative embodiment. The monitoring device 500 may be utilized as an optional subsystem of an LTEExD. The monitoring device 500 may comprise an Absolute Orientation Sensor 502, a signal analyzer 504, a transmitter 508, two electrodes 510, 512, an antenna 514, and a power supply unit 516.

As shown, in one embodiment, the monitoring device 500 and transmitter 508 may be packaged in a single printed circuit board with a single power supply 516. The chest belt electrodes 510, 512 may be placed in electrical communication with the skin and the intravascular blood beneath in order to obtain an electrocardiogram signal.

A well-known existing problem with typical usage of an exercise chest belt sub-system is the need or requirement to moisten the plastic or fabric heart rate sensor electrodes prior to use. To combat dry skin at the beginning of an exercise session, moisture is added to ensure better contact and adequate functioning of the device. When sweating commences, plastic sensor contacts will improve because the salt in sweat begins conducting the electrical signal. If the strap has fabric electrodes, it is essential that the sensors are moistened thoroughly with water before exercise.

Regardless of the need to moisten the sensors, any chest belt sub-system needs appropriate tightening of the elastic strap. If the strap is loose, the movement of the electrodes will disturb ECG signal detection. For best signal acquisition, the elastic belt is initially placed right under the pectoral muscles, but may be adjusted so the sensors are placed onto the mid-back to produce high fidelity ECG signals as well.

One final problem is chest hair may weaken electrode sensor contact areas. The best solution to this issue for existing chest belt sub-systems is to shave a small area on the chest wall so the sensors make better contact, resulting in better conductivity and better ECG signal fidelity.

The main problem with existing exercise chest belt electrodes is that they require skin preparation, conduction gel, or sweat during exercise to reduce the sking electrode interface impedance. This problem not uncommonly causes trouble to users, as they are unable to test chest belt function while at rest, prior to exercise. Also, the addition of conduction gels may leave residue on the chest wall skin or cause short circuit between two electrodes in close proximity. Moreover, these aforementioned preparation procedures are time consuming and uncomfortable, since the skin preparation may involve abrasion of the outer skin layer and/or clipping of the chest hair.

In an alternate embodiment, dry foam electrodes within the exercise chest belt sub-system exhibits electrically conductive polymer foam and fabric, and provides strong capacitive behavior at the chest wall and skin interface points. The electrically conductive polymer foam substrate within the dry electrodes fit the chest wall surface and increase the contact area between skin and electrode, and, as a result, reduce the impedance. The foam-skin interface is not only used to reduce the motion force, but also used to increase the fabric-skin contact area when force is applied on the electrode. The foam will also assimilate the motion force, preventing rubbing and sliding of the electrode on the skin, thus simultaneously reducing the motion artifact and skin-electrode interface impedance.

In this alternate embodiment, E103/HART/Polyester and E103/HART/XAC/Polyethylene are both effective rigid ECG electrically conductive polymer foams with open and closed pores, respectively. Dry foam electrodes may also be covered by a conductive fabric. The somewhat rigid edges of the foam slightly scratch and abrade the skin and thus gently reduce the skin impedance. These unique foam properties make the previously standard preparation of shaving chest hair unnecessary. In addition, these dry foam electrodes are much more resistant to motion artifact, such as that typically produced during heavy exercise when using chest belts with plastic or fabric electrodes.

The signal analyzer 504 sub-system may utilize FFT-based spectral analysis to transfer the electrocardiogram signal from the time domain to the frequency domain. The calculated variables may be telemetered 518 to a remote display 506 sub-system on an observer. In one embodiment, the signal analyzer 504 calculates the RR, HR, and HRV, and transmitter 508 transmits the data to the signal analyzers in the LTEExD 520 headband, as well as a smartwatch and smartphone (e.g., remote display 506).

In one embodiment, the antenna 514 may be positioned within the chest belt, which can wrap around or otherwise encircle the chest anatomy to secure the package during heavy exercise. In an alternative embodiment, the antenna 514 may be positioned externally of the package. In addition, an Absolute Orientation Sensor 502 may be placed within the chest belt in order to detect exercise cadence. In some embodiments, transmission may occur over Bluetooth, Wi-Fi, or other suitable wireless standard.

The remote display 506 sub-system may be used to self-guide subjects in improving athletic performance in the field, and obtaining and perceiving their second wind, through visual feedback on a remote display 506. For example, the remote display may display when lactic threshold is reached.

In some embodiments, Lactic threshold may be detected through FFT-based spectral analysis of HRV during heavy exercise. FFT-based spectral analysis of HRV as not been used in the field before. However, exercise causes progressive withdrawal of vagal activity, with key result being decreased fluctuations in both HF-RSA and LF-THM peaks. This phenomenon may be calculated through FFT-based power spectral analysis in the monitoring device 500 by the signal analyzer 504. Therefore, lactic threshold may be determined through HRV spectral analysis by the signal analyzer 504 scanning for near complete withdrawal of the LF-THM and HF-RSA spectral lines during peak exercise.

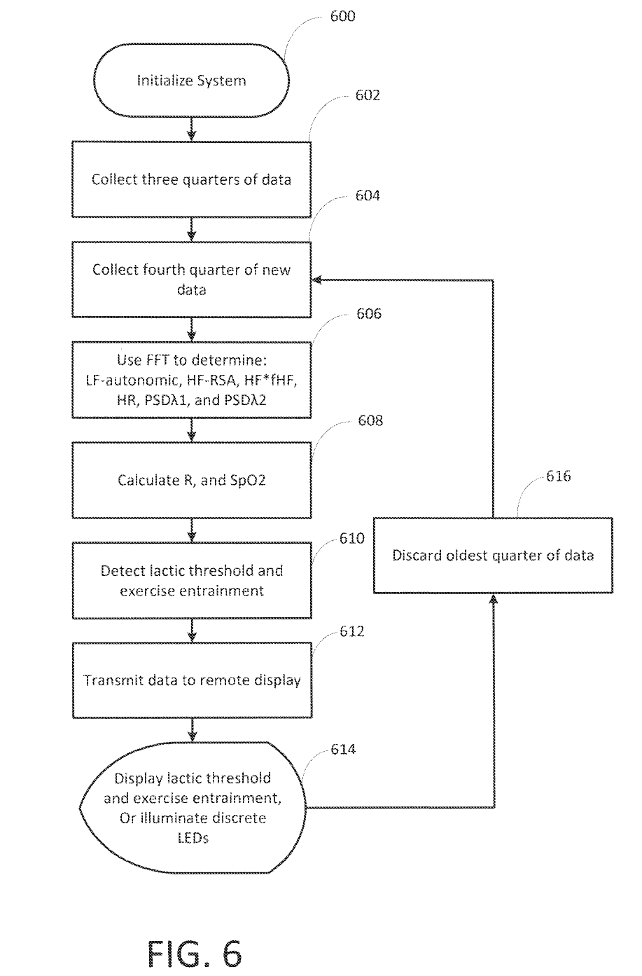

FIG. 6 is a flow diagram illustrating an exemplary process for calculating lactic threshold and entrainment occurrence in accordance with an illustrative embodiment. In some embodiments, microcontroller firmware or computer system software may use these steps to calculate heart rate, HRV, respiratory rate, SpO2, exercise cadence rate, lactic threshold occurrence, and entrainment occurrence.

For example, as explained in more detail below, in one embodiment three-quarters of the data set are kept for processing the next set of calculations by the signal analyzer, with the oldest one-quarter of the data set removed, while the newest one-quarter of the data set is added. The data sets are converted from the time domain to the frequency domain by the fast fourier transform on a microcontroller with floating point unit, and the signal analyzer picks the correct peaks (power spectral density and frequency) for heart rate, HRV, respiratory rate, and exercise cadence, based upon the software algorithm. The signal analyzer utilizes the power spectral data (PSD) and frequency bin data to further calculate the occurrence of lactic threshold and/or entrainment, and displays their yes-no occurrence with discrete red-yellow-green LEDs on a remote display to the observer.

First, a microcontroller may initialize the system, at 200. Such initialization is very system-specific and is well known in the art. After initializing the system, the microcontroller may begin collecting samples of data. A “sample” is the reading of the red and infrared optical intensity values from the PLSIC, three Absolute Orientation Sensor (i.e., readings from the X, Y, and Z axes), and chest belt electrocardiogram beat-to-beat HRV data.

The data may be in various forms. For example, the red and infrared LEDs may be multiplexed, with (1) an intensity value with the red LED emitting (i.e., PSDλ1) and the infrared LED not emitting; and, (2) an intensity value with the IR LED 14 emitting (i.e., PSDλ2) and the red LED not emitting. Further, in one embodiment, the final Absolute Orientation Sensor value may be calculated as the square root of the sum of the squares of the X, Y, and Z axes readings, as is well known in the art.

When either of the LEDs is emitting and a signal is being generated by the interaction of the electromagnetic radiation with the blood, the PLSIC generates a periodic electrical signal in the form of a digital serial pulse train corresponding to the intensity of the optical signal received by the PLSIC. These signals may be interfaced into the microcontroller via I2C and SPI transfer, and an intensity value for the red LED and an intensity value for the IR LED and the final Absolute Orientation Sensor reading may be saved in random access memory (RAM).

Data collection begins at 602. In certain embodiments, the total collection period is 5.69 seconds in this embodiment, which is divided into four quarters of approximately 1.42 seconds each. As shown at 602, three quarters (approximately 4.26 seconds) of data samples may be collected to help initialize a sliding window function. Next, the fourth quarter of the total sample (approximately 1.42 seconds worth of samples) may be taken, at 604. The sample rate and time of collection are all variable, and described here is just one embodiment. In this embodiment, between the samples taken at 602 and 604, a total of 5.69 seconds worth of samples are collected for processing. In some embodiments, the samples may be taken at 720 Hz in order to satisfy the nyquist criteria, as is known in the art, to prevent and minimize aliasing from incandescent and fluorescent light sources.

Based on the data sample, the system may determine the magnitudes of the AC and DC components for both the Red LED and the IR LED (AC.sub.red, DC.sub.red, AC.sub.ir, and DC.sub.ir), the processed Absolute Orientation Sensor value, and the chest belt electrocardiogram data using a frequency domain analysis, at 606. That is, the 5.69 seconds of time-domain data is converted into the frequency domain by performing the well-known Fast Fourier Transform (FFT). The FFT may be performed in many ways, as is known in the art. In one embodiment, an FFT of 4096 points (on data sampled at 720 Hz) will suffice.

For the red and IR optical signals, and the Absolute Orientation Sensor signal, the AC component may be determined by the magnitude of the highest spectral peak found between 0.5 to 3.67 Hz. The two largest peaks commonly represent the pulsatile and exercise cadence components, respectively, of the photo-plethysmogram and Absolute Orientation Sensor waveforms. That is, the frequency bin of the highest Absolute Orientation Sensor spectral line, representing the exercise cadence rate, is discounted in the photo-plethysmogram spectral analysis as a spectral line due to motion artifact. Likewise, the magnitude of the DC component is the highest spectral peak found at 0.0 Hz.

Next, at 608, the program calculates an R value from the red and infrared AC and DC spectral peaks, based on the formula:

R=(AC.sub.red/DC.sub.red)/(AC.sub.ir/DC.sub.ir).

Also, SpO2 is obtained from the approximate formula:

% SpO2=−22.6*R+108.

As described by Arai 1989, in some embodiments the electrocardiogram chest belt data is processed by a signal analyzer, for HRV by low-pass filtering and then sampling with an analog to digital converter at 1000 Hz. The timed occurrence of R-waves of the QRS complex may be detected to 0.001 second accuracy, and the digital beat-to-beat values may be telemetered to a microcontroller, smartwatch, or smartphone. Then, the signal analyzer in the microcontroller, smartwatch, or smartphone may reconstruct the instantaneous (and irregular) beat-to-beat time series data into a regular time series, by filtering out spurious data, and resampling at 4 Hz, in order to construct a regular time series suitable for spectral analysis.

FFT-based power spectral analysis may be performed on the regular time series utilizing 75% overlapping 64-second (×4 Hz=256 points) windowed data sets. In an alternative embodiment, an Auto-Regressive Spectral Estimation method may be utilized in order to improve spectral fidelity, as described by Bolanos 2006. The regular time series may be transformed into the frequency domain, and then heart rate is calculated, respiratory rate is calculated (based upon the HF-RSA frequency bin value), and the LF-THM frequency bins (0.03-0.15 Hz) and HF-RSA frequency bins (0.15 Hz-1.00 Hz) are observed and analyzed as periodograms by the signal analyzer over time for significant changes. In particular, a significant nadir in both the LF-THM and HF-RSA frequency bin intensities is classified by the signal analyzer of the LTEExD as lactic threshold during heavy exercise, as described by Aria 1989 in a controlled indoor exercise laboratory setting.

Additional digital signal processing logic may be used during exercise for lactic threshold and entrainment detection, at 610. For example, in one embodiment, if the Absolute Orientation Sensor peak is within 0.1768 Hz (one frequency bin) of the heart rate peak, entrainment has occurred, and the SpO2 calculation does not occur due to cross-interference with exercise cadence. If there are two large and independent AC peaks in the heart rate frequency bins, the exercise motion peak is identified as the largest one occurring and verified to be the same frequency bin as is occurring in the parallel Absolute Orientation Sensor frequency bin. Similarly, by elimination, the heart rate peak is the other large and independent AC peak that is not the exercise cadence peak. Through appropriate identification of the heart rate peaks in the heart rate frequency bins, an accurate R value and SpO2 calculation may be made, even during heavy exercise. In addition, the exercise cadence component may be calculated from the parallel Absolute Orientation Sensor frequency bin. If electrocardiogram chest belt HRV data is also present, additional verification may be made of the correct heart rate frequency bin in the photo-plethysmogram spectral analysis (that is, versus identification of an motion artifact peak).

Regarding lactic threshold (LT) detection, if SpO2 desaturation occurs during heavy exercise, that is, a delta SpO2 of negative 4% or more from baseline, then lactic threshold has been reached. The LT phenomenon is known to occur in the LTEExD during high intensity interval training (HIIT). Nikooie 2009 described SpO2 desaturation events during heavy exercise, and how the SpO2 vs time inflection point correlates with the occurrence of lactic threshold, by analyzing subjects in an indoor exercise laboratory, in a controlled environment, without benefit of spectral analysis. Analyzing SpO2 desaturation in real-time, in the field, utilizing FFT-based spectral analysis, with a portable and wearable device, is much more difficult, and a significant advancement to the science, and is a key technique of the LTEExD described herein.

Regarding entrainment detection, if the heart rate bins are within one bin (0.1758 Hz) of the exercise cadence bin, then cardio-respiro-locomotor synchronization or entrainment has occurred. This phenomenon is known to occur in the LTEExD during running exercise in high-level professional athletes. O'Rourke 1992 has described entrainment during heavy exercise, and has commented that entrainment has been well known to those skilled in the art since the 1920s, mainly utilizing time-domain based techniques. Entrainment may be confirmed by the specified LTEExD utilizing FFT-based spectral analysis, in real-time, in the field, when respiratory rate, heart rate, and exercise cadence are all integer multiples of one another, without significant remainder. For example, during entrainment, the heart rate may be exactly twice the exercise cadence, and the exercise cadence may be exactly twice the respiratory rate. When the integer factor or multiple rule is met, without significant remainder, then entrainment has occurred, and the result is confirmed and displayed by the described LTEExD on a remote display.

As is known, in the alternative to the FFT, many other methods can be used to determine the AC and DC components of the photo-plethysmogram, Absolute Orientation Sensor, and electrocardiogram HRV data. For example, as is well known in the art, Auto-Regressive (AR) Spectral Estimation may be used for this purpose in the specified LTEExD.

In summary, an LTEExD, may use either of the described AR or FFT spectral analysis methods for analyzing HRV for determination of lactic threshold and/or entrainment. These methods may be applied to either the electrocardiogram chest belt data, or the forehead photo-plethysmogram data, or both, in real-time, in the field, and wirelessly transmitted 612 to a remote display held by an observer (including self).

Next, the calculated heart rate, HRV, arterial hemoglobin oxygen saturation value (SpO2), respiratory rate, exercise cadence, lactic threshold (yes/close/no), and entrainment (yes/close/no) may be displayed at 614 as red/yellow/green on the remote display, or utilizing a similar trivalent indicator system.

Finally, the program loops back to 604, where another one quarter of 5.69 seconds of data is collected. As indicated at 616, the oldest quarter of data is discarded so that 5.69 seconds of data remain (only approximately 1.42 seconds of which is new). Thus a 5.69 second window of data can be thought of as sliding by one-quarter increments, thereby discarding approximately 1.42 seconds of data and sampling a new 1.42 seconds of data. The steps at 604, 606, 608, 610, 612, 614 and 616 are performed repeatedly, thereby displaying a new set of values approximately each 1.42 seconds.

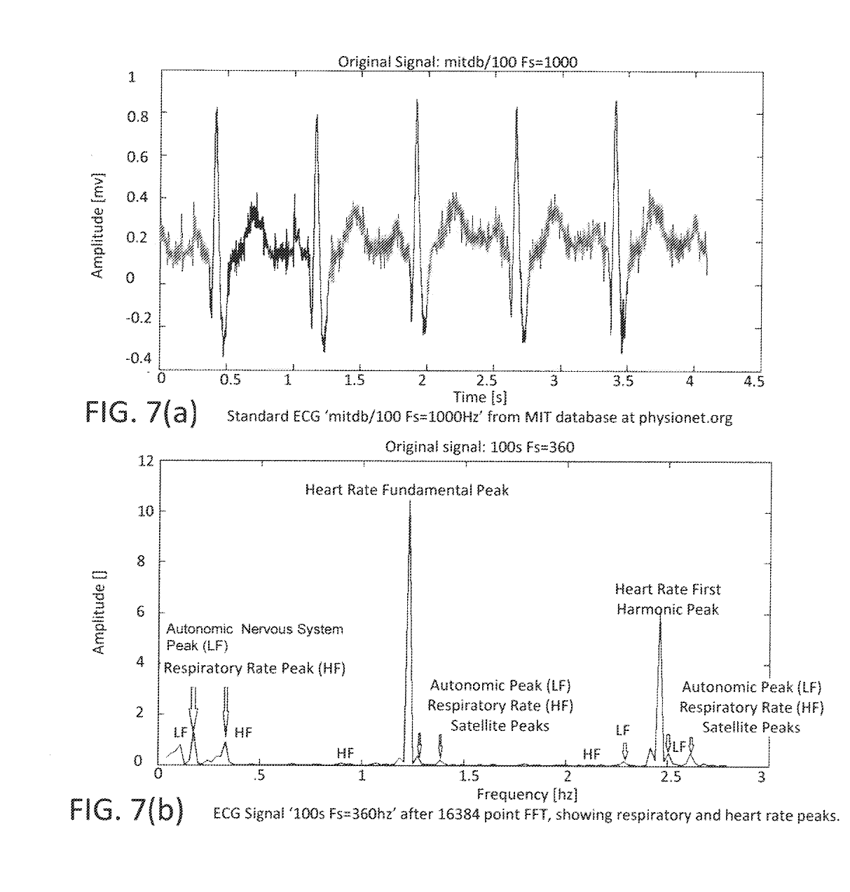

FIG. 7 is an ECG signal used to calculate lactic threshold and exercise entrainment in accordance with an illustrative embodiment. FIG. 7(a) is a Raw ECG High Fidelity Data from MIT Physionet.org database in time domain: name: 100s, sample frequency (Fs)=360 hz. FIG. 7(b) is the same ECG in frequency domain.

The LTEExD device uses an algorithm to calculate Lactic Threshold (LT) and Exercise Entrainment (EE) or cardio-respiro-locomotor synchronization (CRLS) from an ECG as shown. In one embodiment, raw high fidelity electrocardiogram (ECG) data is sampled at 1000 Hz and processed by fast Fourier transform (FFT) in a 4-32 sec rolling window into the frequency domain. Based on analysis of the raw high fidelity ECG data in the frequency domain, the magnitude and frequency (f) of the highest LF-autonomic peak (LF, fLF, 0.04-0.15 hz range) and HF-RSA peak (HF, fHF, 0.15-1.00 Hz range). Note: Respiratory Rate equals fHF. Importantly, LT is noted to occur when a sustained non-linear increase (delta slope (m)>20%) in HF*fHF occurs over a 60 sec time period. In addition, LF energy decreases significantly over the same 60 sec time period as a second and independent check toward LT occurrence. Furthermore, Exercise Entrainment (EE) or cardio-respiro-locomotor synchronization (CRLS) occurs when respiratory rate (e.g. 1:3, 1:4) and cadence rate (e.g. 1:2, 2:3, 1:1) are whole factor numbers of the heart rate.

This new and novel algorithm described above for lactic threshold detection differs significantly from that typically used by Cottin (2007) and/or Polar Electro Oy (U.S. Pat. No. 5,810,722). This older classic algorithm to detect lactic threshold most often utilized by those skilled in the art typically analyzes R-R intervals (that is, not raw high fidelity ECG data, as in the current LTEExD), for example, taken from the Polar Electro Oy (Kempele, Finland) S810-heart rate monitor. The R-R interval (that is, instantaneous heart rate data) time series is resampled at 4-Hz by interpolation of a third order spline function to obtain equidistant data. After resampling this irregularly spaced time series, the RR time series is prefiltered by pass-band finite impulse response (FIR) filters corresponding to HF and LF frequency bands, in order to reduce noise and obtain a merely mono-component signal in each band. A good working example of this algorithm is ‘Kubios HRV—Heart Rate Variability Analysis Software’ (www.kubios.uef.fi). This ‘resampled RR time series method’ has similarly been recommended by the Task Force of the European Society of Cardiology and the North American Society of Pacing and Electrophysiology (1996). However, and most importantly, this algorithm is imperfect and subject to error due to: (i) RR time series being irregularly spaced data, requiring it to be resampled; (ii) prone to motion artifact during heavy exercise and heart beat arrhythmias causing significant interference to the calculations.

The described algorithm may transform a standard ECG into the frequency domain for analysis as shown. For example, FIG. 7(a) shows Raw ECG High Fidelity Data from MIT Physionet.org database in time domain: name: 100s, sample frequency (Fs)=360 hz. FIG. 7(b) is the same ECG in frequency domain. Note LF-autonomic peak at 0.1 hz, HF-RSA peak at 0.3 hz (same as respiratory rate) and Heart Rate peak at 1.2 Hz. For detection of Lactic Threshold (LT) in one embodiment, the HF-RSA peak frequency (HF) and magnitude (fHF) are multiplied together (HF*fHF). LT is detected when a sustained non-linear increase (delta slope (m)>20%) in HF*fHF occurs over a 60 sec time period. Furthermore, Exercise Entrainment (EE) or cardio-respiro-locomotor synchronization (CRLS) occurs when respiratory rate (e.g. 1:3, 1:4) and cadence rate (e.g. 1:2, 2:3, 1:1) are whole factor numbers of the heart rate.

In an alternate embodiment, a two electrode ECG signal acquisition conditioning circuit is improved on and developed using an oversampling method. Oversampling utilizes high speed analog to digital conversion (ADC) to markedly improve the signal-to-noise ratio of the 2-lead ECG signal detected from the exercise chest belt sub-system. This oversampling method is especially important for noise reduction in the LTEExD device where only two electrodes are typically utilized, as a ground lead or a driven-right-leg electrode is typically not included for common mode noise rejection ratio (CMRR) noise reduction.

In this alternative embodiment, the raw high fidelity ECG analog signal is sampled by a high speed ADC at >10 kps (kilosamples per second) in an exercise chest belt sub-system and processed by fast fourier transform (FFT) in a 4-32 sec rolling window into the frequency domain. Based on this ECG spectral analysis, the magnitude and frequency (f) parameters of the highest LF-autonomic peak (LF-magnitude, fLF-hz, 0.04-0.15 hz range) and HF-RSA peak (HF-magnitude, fHF-hz, 0.15-1.00 hz range) are determined. Importantly, respiratory rate equals fHF. Thus, LT is noted to occur, and most reliably and accurately detected, when: (i) a sustained non-linear increase (delta slope-m>20%) in HF*fHF occurs over a 60 sec time period; and, (ii) a sustained non-linear decrease (delta slope-m>20%) in LF-autonomic power occurs over a 60 sec time period; and, (iii) delta instantaneous R-R interval (e.g. R-Ri)<1 msec; and, (iv) delta SpO2>4%. These four parallel algorithms work synergistically together to detect LT; however, one or more of the four algorithms may independently be used to detect LT on its own. Note this algorithm requires ECG sampling at 10+ksps in order to achieve R-Ri resolutions<1 msec, and heretofore has not been attempted nor accomplished in prior art exercise ECG belts, which typically have data sampling rates<=1 ksps, limiting R-Ri resolution>2 msec at most.

In an alternative embodiment, the raw ECG analog signal is oversampled by a high speed ADC at 250 kps (kilosamples per second). Subsequently, once every millisecond, 250 samples are averaged together to produce a final downsampled rate of 1 kps. This oversampled, then downsampled signal is then ready for FFT processing in the MPU and/or CPU sub-systems of the LTEExD device.Ffig.

In an alternative embodiment, the Lomb Periodogram transforms a real-valued time series into a power spectrum using irregularly sampled time series. Importantly, Lomb's method avoids key inaccuracy problems associated with resampling and sample replacement in HRV analysis, especially with missing or noisy ECG data, which is commonly present in exercise field recordings. Thus, Lomb's non-uniform sampling algorithm may be advantageous over more traditional FFT and AR methods for power spectral density estimation in HRV analysis. In addition, Lomb's method utilizing the efficient Press-Rybicki algorithm minimizes computational burden. An alternative embodiment of the novel LTEEx Device fully realizes its digital signal processing capabilities through usage of the Lomb Periodogram to analyze Heart Rate Variability in order to determine Lactic Threshold and Exercise Entrainment variables.

In this alternate embodiment of the novel LTEEx Device, an irregularly sampled instantaneous heart rate (IHR) signal is obtained from the RR interval of the ECG time series. The Lomb algorithm aggressively and advantageously rejects intervals likely to be outliers (whether due to ectopic beats, falsely detected beats, missed beats, or simply mismeasured beat arrival times). When used to derive a power spectral density estimate, Lomb's strategy permits robust derivation of spectra even from highly corrupted time series. In summary, Lomb's power spectral density estimates for the magnitude of autonomic (LF) and respiratory rate (HF) discrete frequency bins allow accurate determination of Lactic Threshold, Respiratory Rate, and Cardio-Respiro-Locomotor Synchronization (CLRS) from ECG signals obtained in the field from exercising subjects.

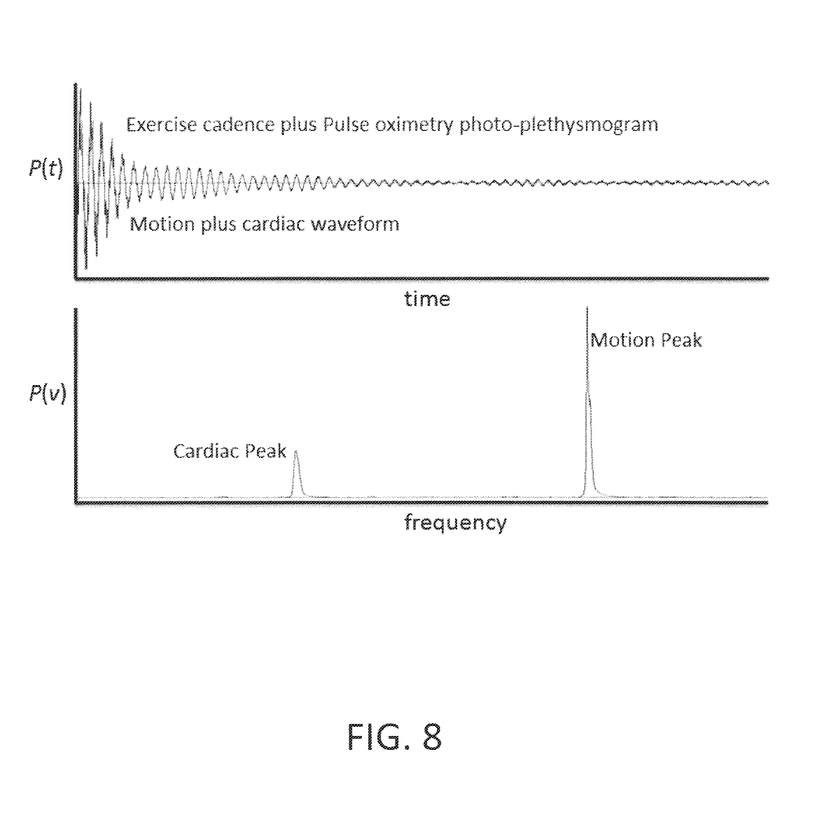

FIG. 8 is a diagram of an FFT-based spectral analysis algorithm of the current LTEExD obtained from a simulated photo-plethysmogram (P(t)) under heavy exercise conditions, with resultant key frequency components and spectral lines (P(v)), including the cardiac peak and the motion peak.

One embodiment of the LTEExD highly utilizes the concepts of the discrete Fourier transform (DFT) and fast Fourier transform (FFT) during heavy exercise. The DFT/FFT converts a finite list of equally spaced samples of a function into a list of coefficients of a finite combination of complex sinusoids, ordered by their frequencies. Fourier analysis can be implemented in computers by numerical algorithms, especially on microcontrollers with dedicated hardware floating point units. The current LTEExD optimizes use of FFT-based spectral analysis by selecting sample rates and window sizes to minimize the problems of aliasing and leakage.

Through the use of FFT-based spectral analysis, the physiologic waveforms (PPG, ECG, and Absolute Orientation Sensor exercise cadence) of the current LTEExD are transformed from time signals into the frequency domain. This approach allows the cardiac frequency and amplitude, respiratory frequency and amplitude, LF-THM frequency and amplitude, HF-RSA frequency and amplitude, and exercise frequency and amplitude to be directly selected from the transform.

FFT-based spectral analysis is particularly advantageous and primary method for digital signal processing for improving SpO2, Heart rate, HRV, Respiratory Rate, and Exercise Cadence accuracies, and decreasing susceptibility to motion artifact and low perfusion errors. The cardiac and cadence signals of interest are sinusoidal signals and are transformed in the FFT as single spectral lines. In addition, the harmonics, noise, and distortion can easily be isolated via fourier analysis. Finally, the LF-THM and HF-RSA frequencies of the autonomic nervous system can be easily isolated as well by using a long enough sample period and by selecting a window size to allow enough frequency bin resolution.

FFT-based spectral analysis is shown to be a practical solution. In one embodiment, the FFT is implemented on a microcontroller with hardware floating point unit for full spectrum analysis, including frequency bins related to physiologic, exercise motion, and optical/electrical waves: LF-THM 0.03-0.15 Hz, HF-RSA 0.15 Hz-1.00 Hz (and respiratory rate), heart rate 0.6-3.7 Hz, exercise cadence 0.5-4.0 Hz, and electrical/optical interference 50/60/120 Hz. In addition, FFT analysis improves accuracy, decreases susceptibility to motion artifact, and improves low perfusion signal analysis. In summary, FFT-based spectral analysis improves accuracy in the LTEExD for the calculated heart rate, HRV, SpO2, respiratory rate, exercise cadence, lactic threshold, and entrainment variables.

Garde 2014 and Shelley 2006 describe the extensive effects of respiration on the photo-plethysmogram (PPG) pulse oximetry waveform in clinical subjects at rest. The first effect is a major shift in the baseline or DC value of the PPG with each breath; the second effect is a change in the amplitude of the PPG with each heartbeat, based upon overall volume status of the intravascular system; and the third effect is a variation in heart rate due to autonomic response to respiration, known as respiratory sinus arrhythmia (RSA). All three of these effects are detected, compared, and quantitated to calculate respiratory rate through FFT-based spectral analysis of the PPG during heavy exercise for the first time in the current LTEExD. In addition, RSA is analyzed through FFT-based spectral analysis to calculate respiratory rate from the electrocardiogram (ECG) based chest belt data stream.

In an alternative embodiment of the current LTEExD, autoregressive (AR) spectral estimation may be utilized for analysis of HRV in PPG and ECG signals during heavy exercise. The AR method is particularly effective for spectral analysis of irregularly sampled HRV data. The autoregressive model specifies that the output variable depends linearly on its own previous values. An estimator applies the Burg algorithm for autoregressive spectral estimation to unevenly spaced data. This method results in much smoother HRV spectral analysis in the LF-THM (0.03-0.15 Hz) and HF-RSA (0.15-1.00 Hz) frequency bins. Radaelli 1991, Lu 2009, and Reyes 2012 all describe FFT-based spectral analysis and AR spectral estimation as effective tools for quantitating HRV from both the ECG and PPG waveforms in resting subjects.

Lu 2009 describes HRV in resting subjects as a valuable measure established by analysis of the temporal relationship between successive heartbeats. Conventionally this signal is determined by electrocardiography (ECG). Each R-wave in the ECG is caused by depolarization of the main mass of the ventricular myocardium. In a parallel manner, any discrete event in the cardiac cycle may be repeatedly measured to produce a record of successive heartbeats. On that basis, cyclical oscillations in blood flow, such as seen in the photo-plethysmogram (PPG), may also be used for HRV analysis. The cyclical PPG oscillations drive volumetric and oxygenation changes in the peripheral microvasculature, and are directly driven by left ventricular contractions.

From a practical point of view regarding wearable exercise devices, a reflectance PPG pulse oximeter sensor is mechanically robust, reusable, and comfortable to wear. In contrast, an ECG Chest Belt worn during heavy exercise may be uncomfortable, be contaminated through electromyography signals, and is subject to baseline drift and electrical interference. Nevertheless, in some embodiments, either ECG-based, or PPG-based, HRV spectral analysis, or both, are effective monitoring tools for detection of lactic threshold and entrainment during heavy exercise.

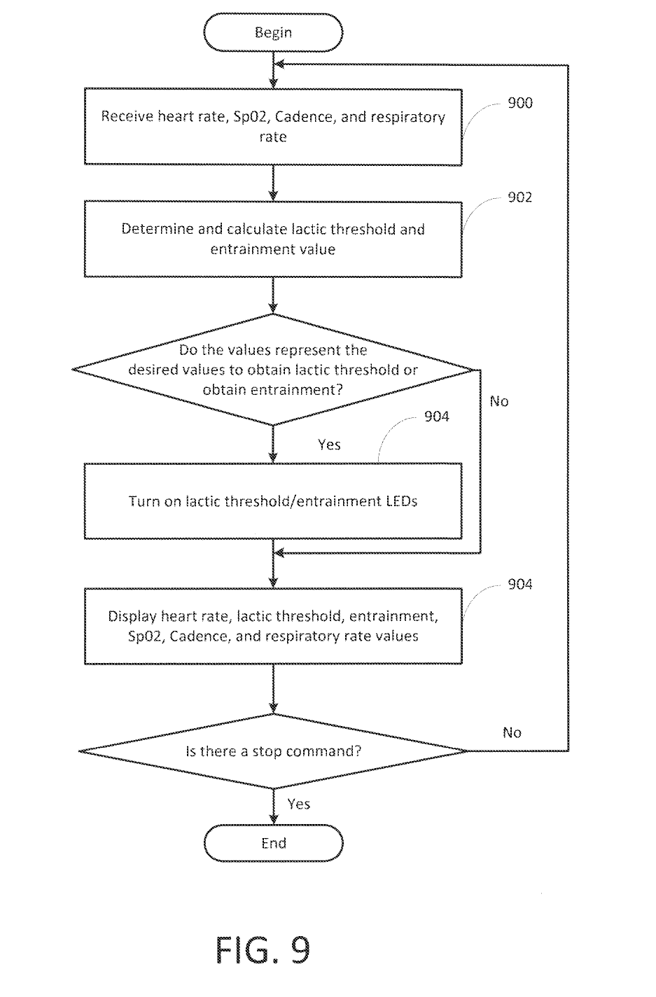

FIG. 9 is a flow chart showing the major process steps taken by a remote display unit to show heart rate, HRV, respiratory rate, SpO2, exercise cadence, lactic threshold, and entrainment to an observer in accordance with an illustrative embodiment.

In one embodiment, the LTEExD may utilize FFT-based spectral analysis to transfer the data from the time domain to the frequency domain. The calculated RPM variable may be telemetered to a remote display sub-system on an observer, along with the heart rate (HR), heart rate variability (HRV), respiratory rate (RR), and SpO2 from a forehead oximeter and/or electrocardiogram chest belt sub-system. For example, the remote display subsystem may receive this data at 900.

This data may be used to determine and calculate a lactic threshold and entrainment value at 902. For example, respiratory rate is entrained by integer-ratio to the cardiac rate and the exercise cadence rate by loco-motor rhythms during heavy exercise. In one embodiment, respiratory sinus arrhythmia (HF-RSA) and respiratory rate are identified by FFT-based spectral analysis of electrocardiogram chest belt or forehead photo-plethysmogram waveforms. The respiratory rate may be compared and divided into the heart rate and exercise cadence, and if an integer ratio has occurred, the signal analyzer in the current LTEExD may identify same integer-based coupling, and alerts the user on a remote display to entrainment occurrence (e.g., Act 904).

In one embodiment, cardio-respiratory-loco-motor entrainment may be measured more precisely and accurately through FFT-based spectral-analysis, in real-time, in the field, in a portable device, with wireless transmission to a remote display (e.g. smartphone or watch).

This may benefit an athlete because entrainment primarily occurs through a hydraulic mechanism, which plays a dominant role in the efficiency of heart function. Integer and phase coupling establishes a feed-forward system of economical co-action, and highly favors functional economy and efficiency. Oxygen uptake is well known to those skilled in the art to be significantly less when entrainment occurs for any given work and power output level, and, in particular, during heavy exercise. Thus, entrainment leads to significant improvements in endurance and speed at the same work and power output.

The remote display sub-unit may self-guide subjects in improving athletic performance in the field, and obtaining and perceiving their second wind, through visual feedback on a remote display (e.g., Act 904 and 906). Feedback may also be given through audio, haptic, or other suitable methods.

In one embodiment, the identification of entrainment may be specified by the LTEExD by alerting the exercising subject on the remote display that entrainment is drawing near or already present. This visual alert (e.g. Red-no entrainment; Yellow-entrainment drawing near; Green-entrainment present) occurs on the display when respiratory rate, heart rate, and cadence rate are integer factors of one another. Visual alerts on a remote display for entrainment may lead to improved competitiveness when running long distances (e.g., 5K or greater) or in any other kind of exercise over an extended period of time.

Physiologically speaking, with entrainment, the cardiac cycle is timed to some advantage to deliver blood when the intramuscular back-pressure is minimal. In fact, entrainment may provide a feeling of well-being, i.e. a ‘second wind’, during heavy exercise, and simultaneously provides a significant competitive advantage. As evidence of the advantages that entrainment may bring forth to users of the current LTEExD, it is well known to those skilled in the art that the natural stride rate of highly competitive runners is very close to their heart rate under heavy exercise conditions.

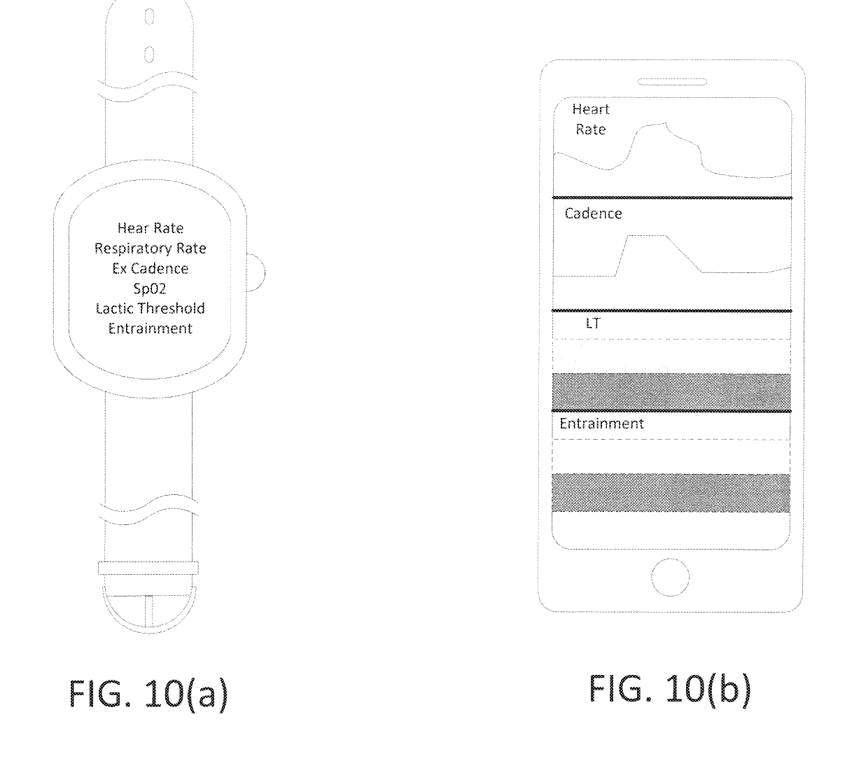

FIG. 10 illustrates two remote display units (smart-watch and smart-phone) in accordance with an illustrative embodiment. Both units have an LCD display with a wireless telemetry module, and are capable of displaying digital values of heart rate, HRV, respiratory rate, SpO2, and exercise cadence.

For example, as FIG. 10(b) demonstrates, the occurrence of lactic threshold and/or entrainment may be represented by red-yellow-green stripes in the display units, or via discrete red-yellow-green LEDs. The observer may optionally see electrocardiogram and pulse oximeter waveforms both in the time and frequency domain as well.

FIG. 10(b) shows example graphs for display on the remote display units heart rate vs time. Additional time vs variable graphs are also available (based on user input) for respiratory rate, SpO2, exercise cadence, lactic threshold, and entrainment.

Display technology for wrist watches and other small devices and smartphones, also well known in the art, provide a very compact and useful low-power remote display 40 during heavy exercise. A suitable smartwatch 120, wearable on the wrist, with suitable display, and Bluetooth 4.0 transceiver, is the Pebble Watch (Pebble Technology, 925. Alma St, Palo Alto, Calif.). A suitable smartphone 200 with a suitable display and Bluetooth 4.0 transceiver is the iPhone 5.0 (Apple, Inc., Cupertino, Calif.). These remote displays may be utilized by the coach or trainer, or self, or by observers and fans alike, in order to follow along and adjust exercise physiology as needed during training or competition.

For example, LT improves with exercise training, and, as a result, moves closer to the maximum metabolic and power output for any given individual (VO2 max). Those who improve LT experience less physical deterioration in muscle cell performance and use less glycogen for ATP production at any level of performance. Thus, improvement in LT through prescribed training allows the athlete to perform at maximal levels for a longer period of time before running out of energy. In essence, an LT-trained athlete with HIIT training under his belt may develop the physical fitness needed to defeat opponents with greater physical strength or determination.

The LT concept has famously led to the thoughtful design of exercise regimens that rapidly improve athletic performance, even at the Olympic and Professional Sports levels. Generally speaking, however, training intensity is not typically prescribed right at LT, but is either much higher or lower intensity. Periodic training at higher intensities than LT is the most valuable training, though is typically limited by coaches since an athlete can quickly over-train in the anaerobic training zone. In turn, assessing the work and power level at LT can be used to evaluate the results of an alternate high and low intensity exercise training program. That is, LT testing is the best marker to evaluate how long training hours are paying off for any athlete willing to wear and use the current LTEExD.

The remote display enables coaches, trainers and athletes to measure both aerobic and anaerobic conditioning by better defining LT. Information about LT is necessary to optimize conditioning, whether the event is 200 Meter Freestyle Swimming or an Ironman Triathlon. With information on each aerobic and anaerobic energy system, a coach may plan, control and monitor the training of athletes with more precision and accuracy. LT data can individualize the intensity of each workout and control training to reach performance objectives in a stepwise process. With LT-based training, there will be no over-training and minimal surprises come race day.

Because lactate is produced by the anaerobic system and used by the aerobic system, it has become a widely recognized and unique marker to measure each system. The amount of energy an athlete can produce per unit of time depends on the development of both aerobic and anaerobic systems, which is why each system is deliberately balanced through training regimens. Essentially, monitoring LT allows for training of the anaerobic system to a level that is appropriate for the athlete's aerobic capacity. This balance will depend upon the event for which the athlete is competing, and will also depend upon the crescendo of the training cycle. In essence, the closer the athlete gets to the “big” race-day event, the more the balance is fine-tuned for peak performance.

Over time, changes in LT reveal what physiological adaptations have taken place. It may tell the coach which forms of training are working or not working. Training time thus becomes much more efficient as the athlete performs only workouts that have benefit. LT becomes the training compass that steers each athlete in the right direction. It is much more relevant than heart rate or power meter monitoring, which typically only reflects a general overall body response to stress. That is, heart rate monitoring and power meter training cannot ever begin to reflect what is actually happening directly in the muscles or within the anaerobic system, that is, in the new way the current LTEExD is able to do.

One way to effectively utilize the LTEExD is by targeting an effort level called maximal lactate steady state (MLSS). MLSS is the maximal level of activity an athlete can continue for an extended period of time, e.g. about an hour, without having to slow down. As long as the athlete maintains this effort level, the blood lactate level will remain constant, typically 4 mmol/L. At small effort levels above this point, lactate level will rise slowly, and the athlete will be forced to stop, sometimes even within a few minutes of the initial rise>4 mmol/L. Above MLSS there are no more steady states, the only option being an inevitable and frequently rapid progression to exhaustion. Training periodically at MLSS improves both sprint and endurance fitness levels dramatically.

With prior art, time and power output at MLSS have been the best indicators of endurance performance. Importantly, prior to the current LTEExD, MLSS could only be verified through blood lactate testing, requiring a finger or ear skin needle prick. The athlete with the best MLSS power will be faster and stronger in an endurance event. Increases in MLSS during training are almost always accompanied by improvements in race performance. For short events, such as swimming and rowing, MLSS is also highly correlated with performance, but, in addition, anaerobic capacity independently becomes more important. The current LTEExD is designed to facilitate non-invasive MLSS power training, no skin needle pricks required.

Unless specifically stated otherwise, as apparent from the following discussions, it may be appreciated that throughout the specification discussions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

In a similar manner, the term “processor” may refer to any device or portion of a device that processes electronic data from registers and/or memory to transform that electronic data into other electronic data that may be stored in registers and/or memory. A “computing platform” may comprise one or more processors.

According to an exemplary embodiment, exemplary methods set forth herein may be performed by an exemplary one or more computer processor(s) adapted to process program logic, which may be embodied on an exemplary computer accessible storage medium, which when such program logic is executed on the exemplary one or more processor(s), may perform such exemplary steps as set forth in the exemplary methods.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

Although the foregoing has included detailed descriptions of some embodiments by way of illustration and example, it will be readily apparent to those of ordinary skill in the art in light of the teachings of these embodiments that numerous changes and modifications may be made without departing from the spirit or scope of the appended claims.

In an illustrative embodiment, any of the operations described herein can be implemented at least in part as computer-readable instructions stored on a computer-readable medium or memory. Upon execution of the computer-readable instructions by a processor, the computer-readable instructions can cause a computing device to perform the operations.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

Unless otherwise defined, each technical or scientific term used herein has the same meaning as commonly understood by one of ordinary skill in the art to which this disclosure belongs. In accordance with the claims that follow and the disclosure provided herein, the following terms are defined with the following meanings, unless explicitly stated otherwise.

The term “about” or “approximately,” when used before a numerical designation or range (e.g., pressure or dimensions), indicates approximations which may vary by (+) or (−) 5%, 1% or 0.1%.

The term “substantially,” when used in the context of substantially eliminating electrical interference, shall mean eliminating at least 80%, at least 90%, at least 95%, or at least 99% of the interference present in a detected signal.

As used in the specification and claims, the singular form “a”, “an”, and “the” include both singular and plural references unless the context clearly dictates otherwise. For example, the term “an evoked potential” may include, and is contemplated to include, a plurality of evoked potentials. At times, the claims and disclosure may include terms such as “a plurality,” “one or more,” or “at least one;” however, the absence of such terms is not intended to mean, and should not be interpreted to mean, that a plurality is not conceived for a particular embodiment.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a device or method consisting essentially of the elements as defined herein would not exclude other materials or steps that do not materially affect the basic and novel characteristic(s) of the claimed invention. “Consisting of” shall mean that the devices, systems, and methods include the recited elements and exclude anything more than a trivial or inconsequential element or step. Embodiments defined by each of these transitional terms are within the scope of this disclosure.

What is claimed is:

1. A portable system for lactic threshold and entrainment detection comprising:
a pulse oximeter configured to measure heart rate and arterial hemoglobin oxygen saturation and produce a corresponding intensity signal;
an accelerometer configured to measure the motion of a user and produce a corresponding motion signal;
a signal analyzer electrically coupled to the pulse oximeter and the accelerometer, wherein the signal analyzer is configured to transform the intensity signal and the motion signal from a time domain into a frequency domain;
one or more processors electrically coupled to the signal analyzer and configured to:
detect heart rate variability in a transformed intensity signal, and exercise cadence in a transformed motion signal;
determine respiratory rate by examining the heart rate variability caused by respiratory sinus arrhythmia; and
compare respiratory rate, heart rate, and exercise cadence to determine whether entrainment has occurred, wherein entrainment is determined to have occurred when respiratory rate, heart rate, and exercise cadence are all integer multiples of one another, without significant remainder; and
a telemetry unit electrically coupled to the one or more processors and configured to transmit to a remote receiver feedback comprising entrainment information to self-guide a subject in obtaining and perceiving a second wind.

2. The portable system if claim 1, wherein:
the one or more processors are further configured to determine whether lactic threshold has been reached by analyzing arterial hemoglobin oxygen saturation, and generate a lactic threshold evaluation based on the lactic threshold determination; and
the feedback transmitted by the telemetry unit further comprises the lactic threshold evaluation.

3. The portable system of claim 2, wherein lactic threshold is determined to have been reached when the arterial hemoglobin oxygen saturation incurs an inflection point.

4. The portable system of claim 3, wherein the inflection point is incurred when there is at least a 4% desaturation in arterial hemoglobin oxygen.

5. The portable system of claim 2, wherein the lactic threshold evaluation feedback is configured for high intensity interval training.

6. The portable system of claim 1, further comprising an electrocardiogram belt comprising:
a second signal analyzer;
a second accelerometer electrically coupled to the second signal analyzer;
at least two electrodes electrically coupled to the signal analyzer, wherein the signal analyzer obtains a raw electrocardiogram high fidelity signal from the electrodes;
a second telemetry unit electrically coupled to the second signal analyzer, wherein the second telemetry unit wirelessly transmits signals obtained by the second signal analyzer from the electrodes and accelerometer to the telemetry unit electrically coupled to the one or more processors.

7. The portable system of claim 6, wherein the second telemetry unit also wirelessly transmits the signals to the remote receiver.

8. The portable system of claim 6, wherein the signal analyzer utilizes a fast Fourier transform based spectral analysis to transform the electrocardiogram signal from the time domain to the frequency domain.

9. The portable system of claim 8, wherein one or more processors are also configured to compare the transformed electrocardiogram signal with the transformed intensity signal to ensure the accuracy of the signals.

10. The portable system of claim 1, wherein the entrainment information feedback provides an indication of how close the subject is to obtaining entrainment.

11. A method comprising:
receiving a heart rate signal, an arterial hemoglobin oxygen saturation value, and a motion signal;
transforming the heart rate and motion signal from a time domain into a frequency domain;
detecting heart rate variability in a transformed heart rate signal;
determining respiratory rate by examining the heart rate variability caused by respiratory sinus arrhythmia;
detecting exercise cadence in a transformed motion signal;
comparing respiratory rate, heart rate, and exercise cadence to determine whether entrainment has occurred, wherein entrainment is determined to have occurred when respiratory rate, heart rate, and exercise cadence are all integer multiples of one another, without significant remainder; and
transmitting to a remote receiver feedback comprising entrainment information to self-guide subjects in obtaining and perceiving a second wind.

12. The method of claim 11, further comprising:
determining whether lactic threshold has been reached by analyzing arterial hemoglobin oxygen saturation;
generating a lactic threshold evaluation based on the lactic threshold determination; and
transmitting the lactic threshold evaluation.

13. The method of claim 12, wherein the lactic threshold evaluation feedback is configured for high intensity interval training.

14. The method of claim 12,
wherein the lactic threshold evaluation assesses whether maximal lactate steady state has been reached, wherein maximal lactate steady state has been reached when lactate levels approach but do not crossing the lactic threshold; and
wherein the lactic threshold evaluation provides instructions to guide a subject in obtaining and maintaining maximal lactate steady state.

15. The method of claim 11, wherein the heart rate signal is received is a raw electrocardiogram high fidelity signal, and further comprising utilizing a fast Fourier transform based spectral analysis to transform the electrocardiogram signal from the time domain to the frequency domain

16. The method of claim 15, wherein determining whether entrainment occurred and lactic threshold has been reached is done using the electrocardiogram signal and a pulse oximeter signal.

17. The method of claim 11, wherein the entrainment information feedback provides an indication of how close the subject is to obtaining entrainment.

18. A method for high intensity interval training comprising:
Receiving, from a pulse oximeter, an arterial hemoglobin oxygen saturation value;
determining, via a processor, whether a user has entered into the anaerobic training zone by analyzing arterial hemoglobin oxygen saturation;
monitoring the time spent in the anaerobic training zone;
transmitting, via a telemetry unit, instructions for a user to decrease training activity to enter in the aerobic training zone after a designated time in spent training in the anaerobic training zone;
determining, via a processor, whether a user has entered into the aerobic training zone by analyzing arterial hemoglobin oxygen saturation;
monitoring the time spent in the aerobic training zone; and
transmitting, via the telemetry unit, instructions for a user to increase training activity to enter in the anaerobic training zone after a designated time in spent training in the aerobic training zone.

19. The method of claim 18, wherein the designated time spent in the anaerobic training zone and the designated time spent in the aerobic training zone are configured to be altered by a user.

20. The method of claim 18, wherein the designated time spent in the anaerobic training zone and the designated time spent in the aerobic training zone are configured to automatically adjust according to a training program.
